(19) United States
(12) Patent Application Publication
(10) Pub. No.: US 2018/0286276 A1
(43) Pub. Date: Oct. 4, 2018

(54) MOBILE TERMINAL AND OPERATION METHOD THEREOF

(71) Applicant: LG ELECTRONICS INC., Seoul (KR)

(72) Inventors: Jisun LEE, Seoul (KR); Soyeon YIM, Seoul (KR); Eugene MYUNG, Seoul (KR); Jungbin LEE, Seoul (KR)

(73) Assignee: LG ELECTRONICS INC., Seoul (KR)

(21) Appl. No.: 15/759,103

(22) PCT Filed: Oct. 5, 2015
(86) PCT No.: PCT/KR2015/010513

§ 371 Date: Mar. 9, 2018

(30) Foreign Application Priority Data
Sep. 11, 2015 (KR) 10-2015-0129180

(51) Int. Cl. G09B 19/00; H04M 1/725; G16H 20/60; G06K 9/62; G06K 9/00

(52) U.S. Cl. CPC G09B 19/0092; H04M 1/72555; G16H 20/60; G06K 9/6293; G06F 3/04883; H04M 2250/52; H04M 2250/74; G06K 2209/17; G06K 9/00671

(57) ABSTRACT

An operation method of a mobile terminal according to an embodiment of the present invention comprises the steps of displaying a first preview image including a food item through a camera provided in the mobile terminal; receiving an input designating an area occupied by the food item; obtaining calories of the food item in response to the received input; and displaying the obtained calories of the food item on one side of the food item.

### TECHNICAL FIELD

The present invention relates to a mobile terminal and a method of operating the same.

### BACKGROUND ART

Terminals may be classified into mobile/portable terminals and stationary terminals depending on the mobility. The mobile terminals may be classified into handheld terminals and vehicle mount terminals according to direct portability by a user.

Functions of the mobile terminals become diversified. The functions of the mobile terminals include data and voice communication, picture capturing and video recording through a camera, voice recording, a music file playback through a speaker system, and image or video output to a display unit. Some terminals further include an electronic game play function or perform a multimedia player function. In particular, recent mobile terminals may receive multicast signals providing visual content such as broadcasts and video or television programs.

According to diversification of functions, such a terminal is implemented in a multimedia player type having complex functions therein, for example, image or video capturing, playback of a music or video file, a game, and broadcast reception.

Recently, technology for grasping eating habits of a user using a terminal has appeared. That is, a food may be recognized through a camera of a terminal and the calorie of the recognized food may be measured.

However, the measured calorie is only an average calorie of the recognized food and may be slightly different from the calorie of the food actually ingested by the user. Therefore, it is difficult to accurately grasp the eating habits of the user.

### DISCLOSURE

### Technical Problem

An object of the present invention is to solve the above-described problems and other problems.

Another object of the present invention is to specifically calculate and store calorie of a food ingested by a user such that the user properly grasps eating habits thereof.

Another object of the present invention is to designate a food to be ingested by a user, to acquire calorie of the designated food and to grasp the calorie of the food ingested by the user in real time.

### Technical Solution

A method of operating a mobile terminal according to one embodiment of the present invention includes displaying a first preview image including a food item through a camera provided in the mobile terminal, receiving input of designating an area occupied by the food item, acquiring a calorie of the food item in response to the received input, and displaying the acquired calorie of the food item at one side of the food item.

A mobile terminal according to one embodiment of the present invention includes a camera, a display unit for displaying a first preview image including a food item acquired through the camera, and a controller for receiving input of designating an area occupied by the food item, acquiring a calorie of the food item in response to the received input, and displaying the acquired calorie of the food item at one side of the food item.

The controller may control the camera and the display unit to display the first preview image when a food intake situation of a user is sensed.

The sensing of the food intake situation may include any one of sensing of smell of a food through a smell sensor provided in the mobile terminal, input of a specific voice command or sound related to food intake through a microphone provided in the mobile terminal, and determining that the mobile terminal is located at a restaurant.

The controller may display a second preview image when end of the food intake situation of the user is sensed and acquires a calorie of the remaining amount of the food item based on the remaining amount of the food item included in the second preview image.

The controller may acquire a calorie ingested by the user using a difference between the calorie of the food item acquired in response to the received input and the calorie of the remaining amount of the food item, and display the calorie ingested by the user.

The controller may output a popup window for providing an eating-habit management service when the food intake situation is sensed.

The controller may recognize a food item included in the first preview image and a food estimation item estimated as a food and acquire information on the recognized food item and information on the food estimation item.

The controller may distinguishably display the food item and the food estimation item.

### Advantageous Effects

According to various embodiments of the present invention, it is possible to specifically calculate and store calorie of a food ingested by a user such to accurately and easily grasps eating habits of the user.

According to various embodiments of the present invention, it is possible to designate a food to be ingested by a user, to acquire calorie of the designated food and to grasp the calorie of the food ingested by the user in real time.

### DESCRIPTION OF DRAWINGS

FIG. 1 is a block diagram for explaining a mobile terminal related to the present invention.

FIG. 2 is a flowchart illustrating a method of operating a mobile terminal according to an embodiment of the present invention.

FIG. 3 is a diagram illustrating an example of displaying an eating-habit management notice according to an embodiment of the present invention.

FIGS. 4a to 4c are diagrams illustrating a process of recognizing a food item through food area designation input within a first preview image according to an embodiment of the present invention.

FIGS. 5a to 5d are diagrams illustrating a process of automatically food items included in a first preview image according to an embodiment of the present invention.

FIGS. 6a to 6e are diagrams illustrating a process of manually recognizing a food included in a first preview image to perform calorie measurement according to another embodiment of the present invention.

FIGS. 7a to 7d are diagrams illustrating a memo function for manually inputting additional information with respect to a recognized food according to an embodiment of the present invention.

FIGS. 8a and 8b are diagrams illustrating an example of an eating-habit management notice according to another embodiment of the present invention.

FIGS. 9a to 9c are diagrams illustrating a process of providing a calorie ingested by a user after the food intake situation of the user ends according to an embodiment of the present invention.

FIGS. 10a to 10d are diagrams illustrating a process of automatically adding calorie information upon sharing a captured food image according to an embodiment of the present invention.

FIGS. 11a to 11d are diagrams illustrating an embodiment of providing a calorie ingested by a user with the passage of time.

FIGS. 12a to 12e are diagrams illustrating a process of guiding the amount of foods to be ingested such that the daily calorie intake amount of a user does not exceed a recommended calorie intake amount according to an embodiment of the present invention.

FIGS. 13a to 13d are diagrams illustrating a process of providing information on foods and calories ingested by a user every day at a glance according to an embodiment of the present invention.

### BEST MODE

Reference will now be made in detail to the embodiments of the present disclosure, examples of which are illustrated in the accompanying drawings, in which like numbers refer to like elements throughout, and a repetitive explanation will be omitted. In the following description, usage of suffixes such as ‘module’, ‘part’ or ‘unit’ used for referring to elements is given merely to facilitate explanation of the present invention, without having any significant meaning by itself. In the following description, detailed descriptions of well-known functions or constructions will be omitted since they would obscure the invention in unnecessary detail. In addition, the accompanying drawings are used to help easily understand the technical idea of the present invention and it should be understood that the idea of the present invention is not limited by the accompanying drawings. However, this invention should not be construed as limited to specific disclosure forms, and the spirit and scope of the invention should be understood as incorporating various modifications, equivalents and substitutions.

It will be understood that, although the terms first, second, etc., may be used herein to distinguish one element from another element, not to be limited by the terms.

It will be understood that when an element is referred to as being “connected” or “coupled” to another element, it may be directly connected or coupled to the other element or intervening elements may be present. In contrast, when an element is referred to as being “directly connected” or “directly coupled” to another element, there are no intervening elements present.

As used herein, the singular forms “a,” “an” and “the” are intended to include the plural forms as well, unless the context clearly indicates otherwise.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

A mobile terminal described herein may include a mobile phone, a smart phone, a laptop computer, a digital broadcast terminal, a personal digital assistants (PDA), a portable multimedia player (PMP), a navigator, a slate PC, a tablet PC, an ultrabook, a wearable device (for example, a smart watch, a smart glass, or a head mounted display (HMD)).

However, those skilled in the art may easily understand that a configuration according to embodiments described herein may also be applied a stationary terminal such as a digital TV, a desktop computer, or a digital signage, except a case of being applied only to a mobile terminal.

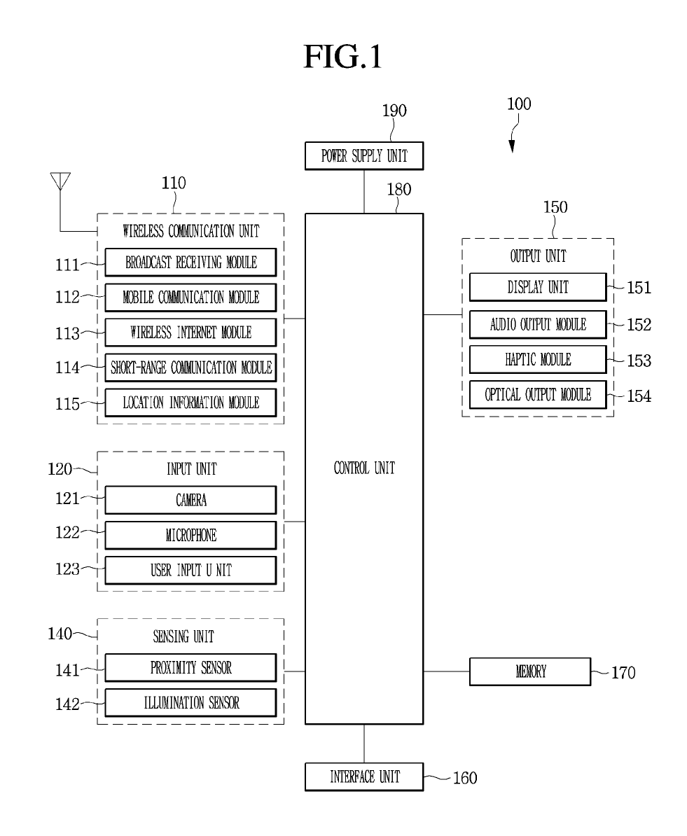

FIG. 1 is a block diagram for explaining a mobile terminal in relation to an embodiment.

The mobile terminal 100 may include a wireless communication unit 110, an input unit 120, a sensing unit 140, an output unit 150, an interface unit 160, a memory unit 170, a control unit 180, and a power supply unit 190. Since the elements illustrated in FIG. 1 are not essential for realizing a mobile terminal, a mobile terminal to be described herein may include more or fewer elements than the above-described.

In detail, the wireless communication unit 110 among the elements may include one or more modules enabling wireless communication between the mobile terminal 100 and another mobile terminal 100, or between the mobile terminal 100 and an external server. In addition, the wireless communication 110 may include one or more modules connecting the mobile terminal 100 to one or more networks.

The wireless communication unit 110 may include at least one of a broadcast reception module 111, a mobile communication module 112, a wireless internet module 113, a short range communication module 114, and a location information module 115.

The input unit 130 may include a camera 121 or an image input unit for an image signal input, a microphone 122 or an audio input unit for an audio signal input, a user input unit 123 (e.g., a touch key, a mechanical key, etc.) for receiving information from a user. Voice data or image data collected by the input unit 120 may be analyzed and processed with user's control commands.

The sensing unit 140 may include at least one sensor for sensing at least one of surrounding environment information around the mobile terminal and user information. For example, the sensing unit 140 may include at least one selected from a proximity sensor 141, an illumination sensor 142, a touch sensor, an acceleration sensor, a magnetic sensor, a G-sensor, a gyroscope sensor, a motion sensor, an RGB sensor, an infrared (IR) sensor, a finger scan sensor, an ultrasonic sensor, an optical sensor (e.g., the camera (see 121)), a microphone (see 122), a battery gauge, an environmental sensor (e.g., a barometer, a hygrometer, a thermometer, a radiation sensor, a thermal sensor, a gas detection sensor, etc.), a chemical sensor (e.g., an e-nose, a healthcare sensor, a biometric sensor, etc.). Furthermore, the mobile terminal disclosed herein may combine and use information sensed by at least two sensors among those sensors.

The output unit 150 is for generating an output related to sense of sight, sense of hearing, or sense of touch, and may include at least one selected from a display unit 151, an audio output unit 152, a haptic module 153, and a light output unit 154. The display unit 151 may form a mutually layered structure with or be formed into one with a touch sensor, and realize a touch screen. Such a touch screen may not only function as the user input unit 123 providing an input interface between the mobile terminal 100 and the user, but also provide an output interface between the mobile terminal 100 and the user.

The interface unit 160 plays a role of a passage with various kinds of external devices connected to the mobile terminal 100. This interface unit 160 may include at least one selected from a wired/wireless headset port, an external charger port, a wired/wireless data port, a memory card port, a port connecting a device having an identification module prepared therein, an audio input/output (I/O) port, a video input/output (I/O) port, and an earphone port. In the mobile terminal 100, a proper control may be performed on a connected external device in correspondence to connection between the external device and the interface unit 160.

In addition, the memory 170 stores data for supporting various functions of the mobile terminal 100. The memory 170 may store a plurality of application programs or applications driven in the mobile terminal 100, data for operations of the mobile terminal 100, and instructions. At least a part of these application programs may exist in the mobile terminal 100 at the time of release for basic functions (e.g., a call originating or receiving function, a message transmitting and receiving function). Moreover, the application programs are stored in the memory 170 and installed in the mobile terminal 100, and then may be driven to perform operations (or functions) of the mobile terminal by the control unit 180.

The control unit 180 typically controls overall operations of the mobile terminal 100 besides operations related to the application programs. The control unit 180 may provide the user with, or process proper information or functions by processing a signal, data, or information input or output through the above-described elements, or driving the application programs stored in the memory 170.

In addition, the control unit 180 may control at least a part of the elements illustrated in FIG. 1 so as to drive the application programs stored in the memory 170. Furthermore, the control unit 180 may combine at least two elements among the elements included in the mobile terminal 100 and operate the combined.

The power supply unit 190 receives internal or external power under a control of the control unit 180 and supplies the power to each element included in the mobile terminal 100. The power supply unit 190 includes a battery and the battery may be an embedded type battery or a replaceable battery.

At least a part of the elements may operate in cooperation with each other for realizing an operation, control, or control method of the mobile terminal according to various embodiments. In addition, the operation, control, or control method of the mobile terminal may be realized in the mobile terminal by driving at least one application program stored in the memory 170.

Hereinafter, the above-described elements are described in detail with reference to FIG. 1 before describing various embodiments realized through the mobile terminal 100.

Firstly, in the wireless communication unit 110, the broadcast reception module 111 receives a broadcast signal and/or broadcast related information from an external broadcast management server through a broadcast channel. The broadcast channel may include a satellite channel or a terrestrial channel. Two or more broadcast reception modules may be provided to the mobile terminal 100 for simultaneous broadcast reception or broadcast channel switching for at least two broadcast channels.

The mobile communication module 112 may transmit and receive wireless signals to and from at least one selected from a base station, an external terminal, and a server on a mobile communication network constructed according to technical standards or communication schemes for the mobile communication (e.g., Global System for Mobile communication (GSM), Code Division Multi Access (CDMA), Code Division Multi Access 2000 (CDMA 2000), Enhanced Voice-Data Optimized or Enhanced Voice-Data Only (EV-DO), Wideband CDMA (WCDMA), High Speed Downlink Packet Access (HSDPA), High Speed Uplink Packet Access (HSUPA), Long Term Evolution (LTE), and Long Term Evolution-Advanced (LTE-A) etc.).

The wireless signal may include a voice call signal, a video call signal, or various types of data according to transmission and reception of a text/multimedia message.

The wireless internet module 113 refers to a module for a wireless internet connection, and may be embedded in or prepared outside the mobile terminal 100. The wireless internet module 113 is configured to transmit and receive a wireless signal over a communication network conforming with wireless internet technologies.

The wireless internet technologies include, for example, Wireless LAN (WLAN), Wireless-Fidelity (Wi-Fi), Wi-Fi Direct, Digital Living Network Alliance (DLNA), Wireless Broadband (WiBro), World Interoperability for Microwave Access (WiMAX), High Speed Downlink Packet Access (HSDPA), High Speed Uplink Packet Access (HSUPA), Long Term Evolution (LTE), and LTE-Advanced (LTE-A), and the wireless internet module 113 transmits and receives data according to at least one wireless internet technology within the range of including internet technology not described in the above.

From a viewpoint that an access to the wireless internet through WiBro, HSDPA, HSUPA, GSM, CDMA, WCDMA, LTE, or LTE-A is conducted through a mobile communication network, the wireless internet module 113 conducting the access to the wireless internet through the mobile communication network may be understood as a kind of the mobile communication module 112.

The short range communication module 114 is for short range communication and may support the short range communication by using at least one selected from Bluetooth™, Radio Frequency Identification (RFID), Infrared Data Association (IrDA), Ultra Wideband UWB), ZigBee, Near Field Communication (NFC), Wi-Fi, Wi-Fi Direct, and Wireless Universal Serial Bus (Wireless USB) technologies. This short range communication module 114 may support, through a wireless area network, wireless communication between the mobile communication terminal 100 and a wireless communication system, between the mobile terminal 100 and another mobile terminal 100, or between the mobile terminal 100 and a network on which the other mobile terminal 100 or an external server is located. The wireless area network may be a wireless personal area network.

Here, the other mobile terminal 100 may be a wearable device (e.g., a smart watch, a smart glass, or an HMD) through which data is mutually exchangeable (or interworkable) with the mobile terminal 100 according to an embodiment. The short range communication module 114 may detect (or recognize) a wearable device capable of communicating with the mobile terminal 100. Furthermore, when the detected wearable device is authenticated to communicate with the mobile terminal 100, the control unit 180 may transmit at least a part of data processed in the mobile terminal 100 to the wearable device through the short range communication module 114. Therefore, a user of the wearable device may use the data processed by the mobile terminal 100 through the wearable device. For example, when a call is received by the mobile terminal 100, the user may perform a phone call through the wearable device, or when a message is received by the mobile terminal 100, the user may check the received message through the wearable device.

The location information module 115 is for obtaining a location (or a current location) of the mobile terminal. As a representative example thereof, there is a global positioning system (GPS) module or a Wi-Fi module. For example, when adopting the GPS module, the mobile terminal may obtain a location of the mobile terminal by using a signal transmitted from a GPS satellite. For another example, when adopting the Wi-Fi module, the mobile terminal may obtain the location of the mobile terminal on the basis of information on a wireless access point (AP) transmitting or receiving a wireless signal with the Wi-Fi module. If necessary, the location information module 115 may additionally or alternatively perform any one function among other modules in the wireless communication unit 110 in order to obtain data about the location of the mobile terminal. The location information module 115 is a module used for obtaining the location (or current location) of the mobile terminal, and is not limited to a module directly calculating or obtaining the location of the mobile terminal.

Next, the input unit 120 is for receiving image information (or an image signal), audio information (or an audio signal), data, or information input from the user. The mobile terminal 100 may include one or a plurality of cameras 121 for an input of image information. The camera 121 processes an image frame such as a still image or video obtained by an image sensor in a video call mode or an image capturing mode. The processed image frame may be displayed on the display unit 151 or stored in the memory 170. Furthermore, the plurality of cameras 121 prepared in the mobile terminal 100 may be arranged to form a matrix structure, and, through the cameras 121 forming this matrix structure, a plurality of pieces of information on images having different angles or different focuses may be input to the mobile terminal 100. In addition, the plurality of cameras 121 may be arranged in a stereo structure to obtain left and right images for realizing a stereoscopic image.

The microphone 122 may process an external sound signal as electrical voice data. The processed voice data may be variously used according to a function (or an application program) being performed in the mobile terminal 100. Furthermore, various noise removal algorithms may be implemented for removing noise generated in a process for receiving the external sound signal.

The user input unit 123 is for receiving information from the user. When information is input through the user input unit 123, the control unit 180 may control an operation of the mobile terminal 100 in correspondence to the input information. This user input unit 123 may include a mechanical input unit (or mechanical key, for example, buttons positioned on the front and rear surfaces or on the side surfaces, a dome switch, a jog wheel, or a jog switch, etc.) and a touch type input unit. As an example, the touch type input unit may be configured with a virtual key displayed on a touch screen through a software processing, a soft key, or a visual key, or a touch key disposed on a portion other than the touch screen. In addition, the virtual key or the visual key is possibly displayed on the touch screen in various types and, for example, may be configured with graphics, texts, icons, videos, or a combination thereof.

Furthermore, the sensing unit 140 may sense at least one of environmental information surrounding the mobile terminal 100 and user information, and generate a sensing signal corresponding to the sensed information. The control unit 180 may control driving or operations of the mobile terminal 100, or perform data processing, a function, or an operation related to an application program installed in the mobile terminal 100, on the basis of the sensing signal. Hereinafter, representative sensors among various sensors that may be included in the sensing unit 140 are described in detail.

Firstly, the proximity sensor 141 refers to a sensor detecting presence of an object accessing or around a predetermined detecting surface by using an electromagnetic force or an infrared ray without a mechanical contact. This proximity sensor 141 may be disposed in an internal area of the mobile terminal surrounded by the above-described touch screen or around the touch screen.

As an example of the proximity sensor 141, there is a transmissive optoelectronic sensor, a diffuse optoelectronic sensor, a high frequency oscillating proximity sensor, a capacitive proximity sensor, an inductive proximity sensor, or an infrared proximity sensor. When the touch screen is capacitive type, the proximity sensor 141 may be configured to detect an access of an object having conductivity by a change of an electric field according to the access of the object. In this case, the touch screen (or a touch sensor) itself may be classified into a proximity sensor.

Moreover, for convenience of explanation, a behavior that an object is in proximity to the touch screen without contacting the touch screen and is allowed to be recognized as if the object is on the touch screen is referred to as a “proximity touch”. A behavior that an object actually contacts the touch screen is referred to as a “contact touch”. A position at which an object is subject to a proximity touch over the touch screen means a position at which the object vertically corresponds to the touch screen when the object is subject to the proximity touch. The proximity sensor 141 may detect a proximity touch and a proximity touch pattern (e.g., a proximity touch distance, a proximity touch direction, a proximity touch speed, a proximity touch time, a proximity touch position, a proximity touch shift state, etc.). Furthermore, the control unit 180 may process data (or information) corresponding to a proximity touch action and the proximity touch pattern detected through the proximity sensor 141 and, in addition, may output visual information corresponding to the processed data on the touch screen. In addition, the control unit 180 may control the mobile terminal 100 so that different operations or different data (or information) are processed according to whether a touch for an identical point on the touch screen is a proximity touch or a contact touch.

The touch sensor senses a touch (or a touch input) applied to the touch screen (or the display unit 151) by using at least one of various touch schemes including a resistive-film scheme, a capacitive scheme, an infrared ray scheme, an ultrasonic scheme, and a magnetic field scheme.

As an example, the touch sensor may be configured to convert a change in pressure applied to a specific part or a change in capacitance generated at a specific part of the touch screen into an electrical input signal. The touch sensor may be configured to detect a position or an area thereon which is touched by a touch object touching the touch screen, or pressure or capacitance at the time of the touch. Here, the touch object may be an object applying a touch on the touch sensor, for example, a finger, a touch pen, a stylus pen, or a pointer.

In this way, when there is a touch input on the touch sensor, a signal (signals) corresponding thereto is (are) transmitted to a touch controller. The touch controller processes the signal(s) and transmits corresponding data to the control unit 180. Accordingly, the control unit 180 may know which area of the display unit 151 is touched. Here, the touch controller may be a separate element other than the control unit 180, or be the control unit itself.

Furthermore, the control unit 180 may perform different controls or an identical control according to a kind of the touch object, which touches the touch screen (or a touch key prepared other than the touch screen). Whether to perform different controls or an identical control according to a kind of the touch object may be determined according to a current operation state of the mobile terminal 100 or an application program being executed.

The above-described touch sensor and proximity sensor may sense independently or in a combined manner various types of touches on the touch screen, wherein the touches include a short (or a tap) touch, a long touch, a multi-touch, a drag touch, a flick touch, a pinch-in touch, a pinch-out, a swipe touch, and a hovering touch.

The ultrasonic sensor may recognize position information on a touch object by using an ultrasonic wave. The control unit 180 is able to calculate a position of a wave generating source through information sensed by an optical sensor and a plurality of ultrasonic sensors. The position of the wave generating source may be calculated by using a property that a light is very faster than the ultrasonic wave, in other words, a time that a light arrives at an optical sensor is very shorter than a time that an ultrasound wave arrives at an ultrasonic sensor. In detail, the position of the wave generating source may be calculated by using a time difference from a time when an ultrasonic wave arrives with a light considered as a reference signal.

Furthermore, from a view of a configuration of the input unit 120, the camera 121 includes at least one selected from a camera sensor (e.g., a CCD, or a CMOS sensor), a photo sensor (or an image sensor), and a laser sensor.

The camera 121 and the laser sensor may be combined together and sense a touch of the sensing target for a 3-dimensional stereoscopic image. The photo sensor may be stacked on a display element, and this photo sensor scans a movement of the sensing target close to the touch screen. In detail, the photo sensor includes photo diodes and transistors in rows/columns and scans a target mounted on the photo sensor by using an electrical signal changed according to an amount of a light applied to the photo diodes. In other words, the photo sensor performs coordinate calculation on the sensing target according to a change amount of the light and, through this, position information on the sensing target may be obtained.

The display unit 151 displays (outputs) information processed by the mobile terminal 100. For example, the display unit 151 may display execution screen information on the application program driven in the mobile terminal 100 or user interface (UI) information or graphic user interface (GUI) information according to the execution screen information.

In addition, the display unit 151 may be configured as a stereoscopic display unit displaying a stereoscopic image.

A 3-dimensional display scheme such as a stereoscopic scheme (glasses type), an autostereoscopic scheme (glassless type), or a projection scheme (a holographic scheme) may be applied to the stereoscopic display unit.

The sound output unit 152 may output audio data received from the wireless communication unit 110 or stored in the memory 170 in a call signal reception mode, a call mode or a recording mode, a speech recognition mode, or in a broadcast reception mode. The sound output unit 152 may output a sound signal related to a function (e.g., a call signal reception sound, or a message reception sound, etc.) performed in the mobile terminal 100. This sound output unit 152 may include a receiver, a speaker, or a buzzer, etc.

The haptic module 153 may generate various tactile effects that the user may feel. A representative example of the tactile effect that is generated by the haptic module 153 may be vibration. Strength and a pattern of the vibration generated by the haptic module 153 may be controlled by user selection or setting by the control unit. For example, the haptic module 153 may output different vibrations sequentially or by synthesizing them.

Besides the vibration, the haptic module 153 may generate various tactile effects including an effect by a stimulus such as a pin array moving vertically to a contact skin surface, a air discharge force or air absorptive power through an outlet or an inlet, brush against a skin surface, contact to an electrode, or static electricity, and an effect by reproducing a cold and warmth sense by using a device that heat absorption or heating is enabled.

The haptic module 153 may be implemented to transfer the tactile effect through a direct contact, and may also be implemented for the user to feel the tactile effect through a muscle sense of a finger or an arm. The haptic module 153 may be prepared two or more in number according to a configuration aspect of the mobile terminal 100.

The optical output unit 154 may output a signal for notifying an event occurrence by using a light from an optical source of the mobile terminal 100. The event occurred in the mobile terminal 100 may be exemplified with message reception, call signal reception, missed calls, alarm, schedule notification, email reception, or information reception through an application.

The signal output by the optical output unit 154 is implemented according to that the mobile terminal emits a monochromatic light or a multi-chromatic light towards the front or rear surface. The signal output may be completed when the mobile terminal detects that the user checks the event.

The interface unit 160 may play a role of a passage with all external devices connected to the mobile terminal 100. The interface unit 160 may receive data from the external device, receive power and transfer the power to each element inside the mobile terminal 100, or allow internal data of the mobile terminal 100 to be transmitted to the external device. For example, the interface 160 may include a wired/wireless headset port, an external charger port, a wired/wireless data port, a memory card port, a port connecting a device that an identification module is prepared, an audio input/output (I/O) port, a video input/output (I/O) port, or an earphone port, etc.

Furthermore, the identification module is a chip storing various pieces of information for authenticating user's authority for the mobile terminal 100 and may include a user identify module (UIM), a subscriber identity module (SIM), or a universal subscriber identity module (USIM). A device including the identification module (hereinafter, an ‘identification device’) may be manufactured in a smart card type. Accordingly, the identification device may be connected to the mobile terminal 100 through the interface unit 160.

In addition, when the mobile terminal 100 is connected to an external cradle, the interface unit 160 may be a passage through which power is supplied from the cradle to the mobile terminal 100 or a passage through which various command signals input from the cradle by the user are delivered. The various command signals or the power input from the cradle may operate as signals for perceiving that the mobile terminal 100 is accurately mounted in the cradle.

The memory 170 may store a program for operations of the control unit 180 and temporarily store input/output data (e.g., a phone book, messages, still images, videos, etc.). The memory 170 may store data about vibrations of various patterns and sounds at the time of a touch input on the touch screen.

The memory 170 may include at least one storage medium type among a flash memory type, a hard disk type, a Solid State Disk (SSD) type, a Silicon Disk Drive (SDD) type, a multimedia card micro type, a card type memory (e.g., SD or XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk and an optical disc. The mobile terminal 100 may operate in relation to a web storage performing a storage function of the memory 170 over the internet.

Furthermore, as described above, the controller 180 normally controls overall operations and an operation related to an application program of the mobile terminal 100. For example, when a state of the mobile terminal satisfies a set condition, the control unit 180 executes or releases a lock state that limits an input of a user's control command to applications.

In addition, the control unit 180 may perform a control or a process related to a voice call, data communication, or a video call, etc., or may perform a pattern recognition processing for recognizing a written input and a drawing input performed on the touch screen as a character and an image, respectively. Furthermore, the control 180 may combine and control any one of or a plurality of the above-described elements in order to implement various embodiments to be described below in the mobile terminal 100.

The power supply unit 190 receives external or internal power under a control of the control unit 180 and supplies power necessary for operating each element. The power supply unit 190 includes a battery. The battery may be an embedded battery that is rechargeable and may be detachably coupled for charging.

The power supply unit 190 may include a connection port, and the connection port may be configured as an example of the interface 160 to which an external charger providing power is electrically connected for charging the battery.

As another example, the power supply unit 190 may be configured to charge the battery in a wireless manner without using the connection port. In this case, the power supply unit 190 may receive, from an external wireless power transmitting device, power by using one or more of an inductive coupling manner on the basis of a magnetic induction phenomenon and a magnetic resonance coupling manner on the basis of an electromagnetic resonance phenomenon.

Hereinafter, various embodiments may be implemented in a recording medium that is readable with a computer or a similar device by using software, hardware, or a combination thereof.

Next, description is made about a communication system realizable through the mobile terminal 100 according to an embodiment.

Firstly, the communication system may use different wireless interfaces and/or a physical layer. For example, the wireless interface available by the communication system may include Frequency Division Multiple Access (FDMA), Time Division Multiple Access (TDMA), Code Division Multiple Access (CDMA), Universal Mobile Telecommunications Systems (UMTS)(in particular, Long Term Evolution (LTE), or Long Term Evolution-Advanced (LTE-A)), Global System for Mobile Communications (GSM), or etc.

Hereinafter, for convenience of explanation, description is made limitedly to CDMA. However, it is obvious that the embodiments may be applied to all communication systems including an Orthogonal Frequency Division Multiplexing (OFDM) wireless communication system as well as a CDMA wireless communication system.

The CDMA wireless communication system may include at least one terminal 100, at least one base station (BS, also may be referred to as Node B or Evolved Node B), at least one BS controller (BSC) and a mobile switching center (MSC). The MSC may be configured to be connected to the Public Switched Telephone Network (PSTN) and BSCs. The BSCs may be connected to the BS in pair through a backhaul line. The backhaul line may be prepared according to at least one selected from E1/T1, ATM, IP, PPP, Frame Relay, HDSL, ADSL, and xDSL. Accordingly, the plurality of BSCs may be included in a CDMA wireless communication system.

Each of a plurality of BSs may include at least one sector, and each sector may include an omni-directional antenna or an antenna indicating a specific radial direction from the BS. In addition, each sector may include two or more antennas having various types. Each BS may be configured to support a plurality of frequency allocations and each of the plurality of allocated frequencies may have specific spectrum (e.g., 1.25 MHz, or 5 MHz).

An intersection between the sector and the frequency allocation may be called as a CDMA channel. The BS may be called as a base station transceiver subsystem (BTSs). In this case, one BSC and at least one BS are called together as a “base station”. The base station may also represent a “cell site”. In addition, each of a plurality of sectors for a specific BS may also be called as a plurality of cell sites.

A broadcasting transmitter (BT) transmits a broadcast signal to terminals 100 operated in a system. The broadcast reception module 111 illustrated in FIG. 1 is prepared in the terminal 100 for receiving the broadcast signal transmitted by the BT.

Furthermore, in the CDMA wireless communication system, a global positioning system (GPS) may be linked for checking a location of the mobile terminal 100. A satellite is helpful for grasping the location of the mobile terminal. Useful location information may be obtained by less than two or at least two satellites. Here, the location of the mobile terminal 100 may be tracked by using all techniques, which are capable of tracking the location, as well as a GPS tracking technique. In addition, at least one of GPS satellites may be selectively or additionally responsible for transmitting satellite digital multimedia broadcasting (DMB).

The location information module 115 prepared in the mobile terminal 100 is for detecting, operating or identifying the location of the mobile terminal 100, and may representatively include a GPS module and a WiFi module. If necessary, the location information module 115 may alternatively or additionally perform any function of other modules in the wireless communication unit 110 for obtaining data for the location of the mobile terminal 100.

The GPS module 115 may precisely calculate 3D current location information according to latitude, longitude, and altitude by calculating distance information from three or more satellites and precise time information, and by applying a trigonometry to the calculated information. A method is currently widely used that calculates location and time information using three satellites, and corrects an error in the calculated location and time information using another satellite. The GPS module 115 may calculate speed information by continuously calculating a current location in real time. However, it is difficult to precisely measure the location of the mobile terminal 100 by using the GPS module in a dead zone, such as an indoor area, of the satellite signal. Accordingly, in order to compensate for location measurement in the GPS manner, a WiFi positioning system (WPS) may be used.

The WPS is a technique for tracking the location of the mobile terminal 100 using a WiFi module prepared in the mobile terminal 100 and a wireless access point (AP) transmitting or receiving a wireless signal to or from the WiFi module, and may mean a location measurement technique based on a wireless local area network (WLAN) using WiFi.

The WPS may include a WiFi positioning server, the mobile terminal 100, a wireless AP connected to the mobile terminal 100, and a database storing arbitrary wireless AP information.

The mobile terminal 100 connected to the wireless AP may transmit a location information request message to the WiFi positioning server.

The WiFi positioning server extracts information on the wireless AP connected to the mobile terminal 100 on the basis of the location information request message (or a signal) of the mobile terminal 100. The information on the wireless AP connected to the mobile terminal 100 may be transmitted to the WiFi positioning server through the mobile terminal 100 or transmitted to the WiFi positioning server from the wireless AP.

The information on the wireless AP, which is extracted on the basis of the location information request message of the mobile terminal 100, may be at least one selected from a MAC address, a Service Set Identification (SSID), a Received Signal Strength Indicator (RSSI), Reference Signal Received Power (RSRP), Reference Signal Received Quality (RSRQ), channel information, Privacy, a Network Type, Signal Strength, and Noise Strength.

As described above, the WiFi positioning server may receive information on the wireless AP connected to the mobile terminal 100, and extract wireless AP information corresponding to the wireless AP to which the mobile terminal is being connected from the pre-constructed database. At this point, information on arbitrary wireless APs, which is stored in the database, may be information on a MAC Address, an SSID, channel information, Privacy, a Network Type, latitudinal and longitudinal coordinates of a wireless AP, a building name and floor on which the wireless AP is located, indoor detailed location information (GPS coordinates available), an address of an owner of the wireless AP, a phone number, and etc. At this point, in order to remove a wireless AP provided by using a mobile AP or an illegal MAC address in the location measurement process, the WiFi positioning server may extract a predetermined number of pieces of wireless AP information in the descending order of an RSSI.

Thereafter, the WiFi positioning server may extract (or analyze) location information on the mobile terminal 100 by using at least one piece of wireless AP information extracted from the database. The location information of the mobile terminal 100 is extracted (or analyzed) by comparing the stored and the received wireless AP information.

As a method of extracting (or analyzing) location information on the mobile terminal 100, a cell-ID method, a fingerprint method, a trigonometry, and a landmark method may be used.

The cell-ID method is a method of determining a location of a wireless AP having strongest strength from among surrounding wireless AP information collected by a mobile terminal. This method is advantageous in that implementation is simple, an additional cost is not necessary, and location information may be rapidly obtained. However, when installation intensity of a wireless AP is lower, positioning precision becomes lowered.

The fingerprint method is a method of selecting a reference location in a service area, collecting signal strength information, and estimating a location through signal strength information transmitted from a mobile terminal on the basis of the collected information. In order to use the fingerprint method, it is necessary to construct a database for propagation characteristics in advance.

The trigonometry is a method of operating a location of a mobile terminal on the basis of a distance between coordinates of at least three wireless APs and the mobile terminal. For estimating the distances between the mobile terminal and the wireless APs, signal strength is converted into distance information, or a time of arrival (ToA) of a wireless signal, a time difference of arrival (TDoA) of a wireless signal, an angle of arrival (AoA) of a wireless signal may be used.

The landmark method is a method of measuring a location of a mobile terminal by using a landmark transmitter.

Besides the above-described methods, various algorithms may be used for extracting (or analyzing) location information on a mobile terminal.

The location information on the mobile terminal 100 extracted in this way may be transmitted to the mobile terminal 100 through the WiFi positioning server and the mobile terminal 100 may obtain the location information.

The mobile terminal 100 may obtain location information by being connected to at least one wireless AP. At this point, the number of wireless APs requested for obtaining the location information on the mobile terminal 100 may be variously varied according to a wireless communication environment in which the mobile terminal 100 is located.

Next, a method of operating a mobile terminal according to an embodiment of the present invention will be described.

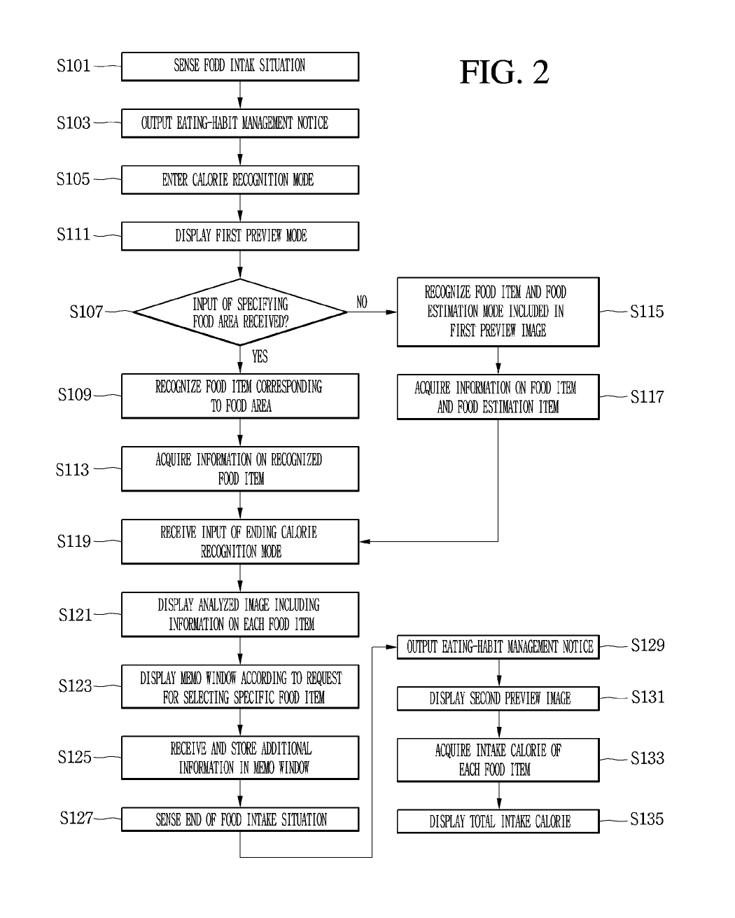

FIG. 2 is a flowchart illustrating a method of operating a mobile terminal according to an embodiment of the present invention.

The control unit 180 of the mobile terminal 100 senses the food intake situation of a user (S101). In one embodiment, the control unit 180 may confirm that the food intake situation is sensed, when the smell of a food is sensed through a smell sensor provided in the sensing unit 140. The smell sensor may be formed of metal-oxide semiconductor to sense smell molecules present in the air around the mobile terminal 100. The control unit 180 may determine whether the user is in a food intake situation using the sensed smell molecules.

In another embodiment, the control unit 180 may sense the food intake situation based on a voice command or sound related to food intake input to the microphone 122. For example, when a voice command <I will enjoy this food or It will be delicious> is received through the microphone 122, the control unit 180 may sense the food intake situation. As another example, when the sound of hitting dishes is input to the microphone 122, a predetermined number of times or more within a predetermined time, the control unit 180 may determine that the user is in the food intake situation.

In another embodiment, the control unit 180 may sense the food intake situation of the user based on the current position of the mobile terminal 100. For example, upon determining that the mobile terminal 100 is located in a restaurant through the location information module 115, the control unit 180 may determine that the user is in the food intake situation.

In another embodiment, when a short-range wireless communication service provided by the restaurant is received, the control unit 180 may determine that the user is in the food intake situation. For example, when the mobile terminal 100 is located in the restaurant and a food menu provided by the restaurant is received from a terminal provided in the restaurant through short-range wireless communication, the control unit 180 may determine that the user is in the food intake situation. Short-range wireless communication may be Bluetooth Low Energy (BLE) communication.

In another embodiment, when a meal start time set by the user arrives, the control unit 180 may determine that the user is in the food intake situation. For example, in the case where a meal time set by the user is 8:30 AM to 9:00 AM, the control unit 180 may determine that the user is in the food intake situation when the current time is 8:30 AM.

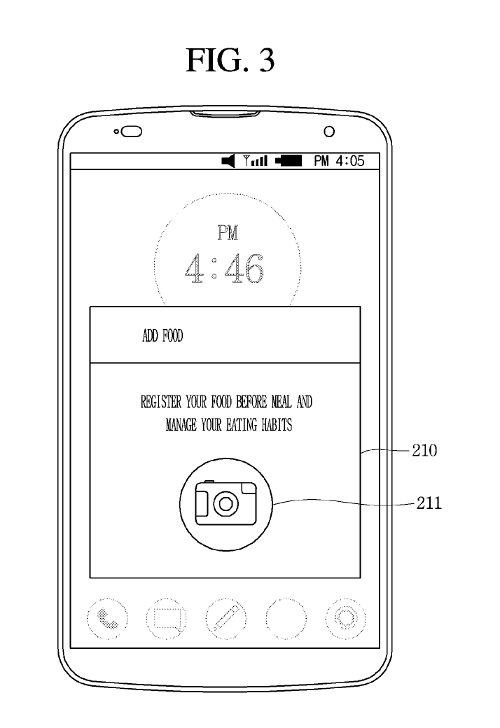

The control unit 180 outputs an eating-habit management notice as the food intake situation is sensed (S103). In one embodiment, the control unit 180 may register foods to be ingested before meal and display the eating-habit management notice for guiding eating-habit management through the display unit 151. This will be described with reference to FIG. 3.

FIG. 3 is a diagram illustrating an example of displaying an eating-habit management notice according to an embodiment of the present invention.

Upon sensing the food intake situation of the user, the control unit 180 of the mobile terminal 100 may display a popup window 210 for providing an eating-habit management notice through the display unit 151. The popup window 210 may include guide text “Register your food before meal and manage your eating habits” and a camera icon 211 having a camera shape. The camera icon 211 may be an icon for switching the operation mode of the mobile terminal 100 to a calorie recognition mode. The calorie recognition mode may refer to a mode for recognizing a food through a captured image and recognizing the calorie of the recognized food. The calorie recognition mode may operate in an image capturing mode.

In another embodiment, the camera icon 211 may be an icon for executing an eating-habit management application. That is, the control unit 180 may execute the eating-habit management application for providing an eating-habit management service according to a request for selecting the camera icon 211. Therefore, the mobile terminal 100 may operate in the calorie recognition mode.

FIG. 2 will be described again.

The control unit 180 switches the operation mode of the mobile terminal 100 to the calorie recognition mode according to the request for the eating-habit management service (S105). In one embodiment, the request for the eating-habit management service may be a request for selecting the camera icon 211 shown in FIG. 3.

In another embodiment, the request for the eating-habit management service may be input of touching a point of the popup window 210 shown in FIG. 3.

In another embodiment, the control unit 180 may switch the operation mode of the mobile terminal 100 to the calorie recognition mode according to a request for selecting an app icon corresponding to the eating-habit management application without performing step S101 of sensing the food intake situation.

In another embodiment, the control unit 180 may switch the operation mode of the mobile terminal 100 to the calorie recognition mode according to a request for selecting a calorie icon displayed on a preview image in the image capture mode. That is, the control unit 180 may operate the mobile terminal 100 in the image capturing mode and operate the mobile terminal in the calorie recognition mode, as the food intake situation is sensed. In this case, steps S101 to S103 may be omitted. This will be described below.

The control unit 180 displays a first preview image through the display unit 151 upon entering the calorie recognition mode (S107). The control unit 180 may power the camera 121 on as the mobile terminal 100 enters the calorie recognition mode and display the first preview image acquired by the camera 121 through the display unit 151.

The first preview image may include a plurality of food items corresponding to a plurality of foods to be ingested.

The control unit 180 determines whether food area designation input of designating a food area in the first preview image is received (S109). In one embodiment, the food area designation input may be input of designating an area occupied by a specific food item among the plurality of food items included in the first preview image. The food area designation input may be touch input of drawing an area occupied by a food through touch of the user.

In another embodiment, the food area designation input may be received on an image obtained by capturing the first preview image.

When the food area designation input is received, the control unit 180 recognizes a food item corresponding to the designated food area (S111) and acquires information on the recognized food item (S113). In one embodiment, the control unit 180 may recognize the type of the food item located in the designated food area using the smell sensor.

In another embodiment, the control unit 180 may capture the food item located in the designated food area, compare the captured food item with the food items stored in the memory 170, and recognize the type of the captured food item. As another example, the control unit 180 may transmit the captured food item to a server linked to the eating-habit management application and receive information on the captured food item from the server, thereby recognizing the food item.

In another embodiment, the control unit 180 may recognize the food item corresponding to the designated food area using an object recognition sensor. The object recognition sensor may be a sensor for emitting a laser to a specific object and recognizing the object using the reflected laser. The control unit 180 may emit a laser to the food item corresponding to the designated food area and recognize the food item using the reflected laser through the object recognition sensor.

In one embodiment, information on the recognized food item may be one or more of the type of the food, the calorie of the food, and the amount (g) of food.

The control unit 180 may recognize the food item and acquire information on the food item. The control unit 180 may display the acquired information on the food item at one side of the food item. Steps S107 to S115 will be described with reference to the following drawings.

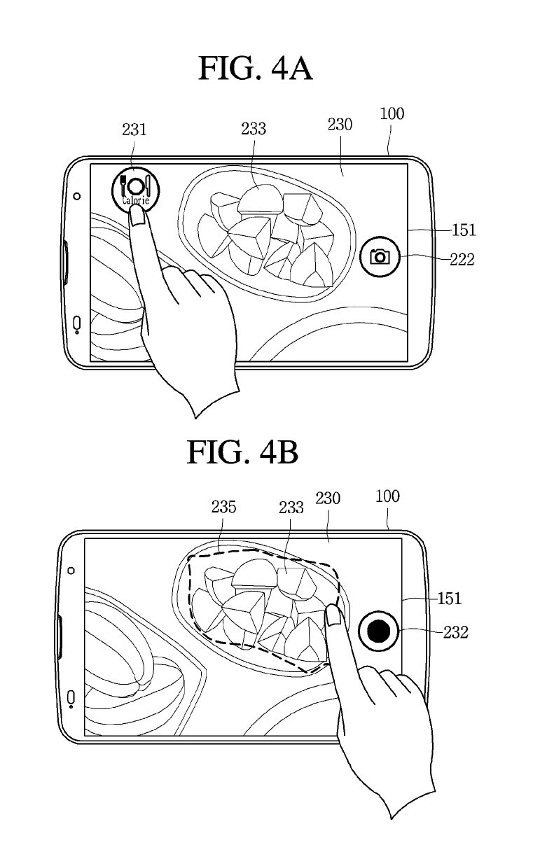

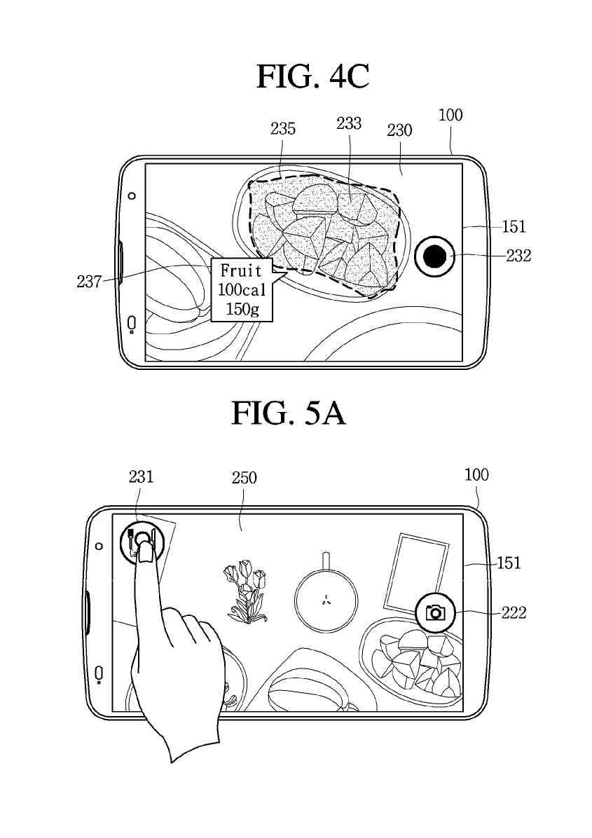

FIGS. 4a to 4c are diagrams illustrating a process of recognizing a food item through food area designation input within a first preview image according to an embodiment of the present invention.

Referring to FIG. 4a, the display unit 151 of the mobile terminal 100 displays the first preview image 230 in the image capturing mode. The first preview image 230 includes a food item 233. According to one embodiment, the mobile terminal 100 may currently operate in the calorie recognition mode. According to another embodiment, the mobile terminal 100 may be in the pre-entry state of the calorie recognition mode. The display unit 151 may display a calorie icon 231 for entering the calorie recognition mode and an image capturing icon 222 for capturing the first preview image 230. The control unit 180 may switch the operation mode of the mobile terminal 100 to the calorie recognition mode according to a request for selecting the calorie icon 231, as shown in FIG. 4b.

Referring to FIG. 4b, if the mobile terminal 100 operates in the calorie recognition mode, the control unit 180 may display an indicator 232 indicating entry into the calorie recognition mode through the display unit 151. That is, the image capturing icon 222 may be switched into the indicator. If the indicator 232 is selected, the indicator 232 may be switched to the image capturing icon. When food area designation input of designating the area occupied by the food item 233 is received, the control unit 180 may display a designation indicator 235 indicating a designated area. The food area designation input may be touch input of drawing a closed curve from one point to another pint along the outline of the area occupied by the food item 233.

The designation indicator 235 may be indicated by a dotted line, but is merely an example and may have any form distinguishable in the first preview image 230. The designation indicator 235 may have a closed curve shape. When food area designation input is received with respect to the food item 233, the control unit 180 may recognize the food item 233 and display information 237 on the recognized food item 233, as shown in FIG. 4c. The information 237 on the food item 233 may include one or more of the type of the food item 233, the calorie of the food item 233 and the amount of the food item.

According to another embodiment of the present invention, when food area designation input is received, the control unit 180 may display a category associated with the food item 233. For example, upon determining that the food item 233 is a meat item through the smell sensor, the control unit 180 may display a category indicating that the food item 233 is meat and display an input popup window capable of inputting the name of the food item. The user may input the name of the food item 233 through the input popup window and register the food item 233 through the memory 170.

FIG. 2 will be described again.

Meanwhile, when food area designation input is not received (S109), the control unit 180 recognizes a food item included in the first preview image and a food estimation item estimated as a food (S115). In one embodiment, the food estimation item may be an item which is recognized as a food but needs to be adjusted to acquire a full image of the food because the image of the food is cut off or overlapped in the first preview image.

In one embodiment, the control unit 180 may automatically recognize the food item included in the first preview image after displaying the first preview image.

In another embodiment, as the first preview image is captured, the control unit 180 may recognize the food item included in the captured first preview image.

The control unit 180 acquires information on the recognized food item and information on the recognized food estimation item (S117). The control unit 180 may recognize the food item and the food estimation item included in the first preview image using one or more of the smell sensor, the object recognition sensor and capturing of the first preview image described in step S113.

The control unit 180 may display the food item and the food estimation item included in the first preview image to be distinguished from the remaining image. In addition, the control unit 180 may display the food item and the food estimation item to be distinguished from each other. Steps S115 to S117 will be described with reference to the following drawings.

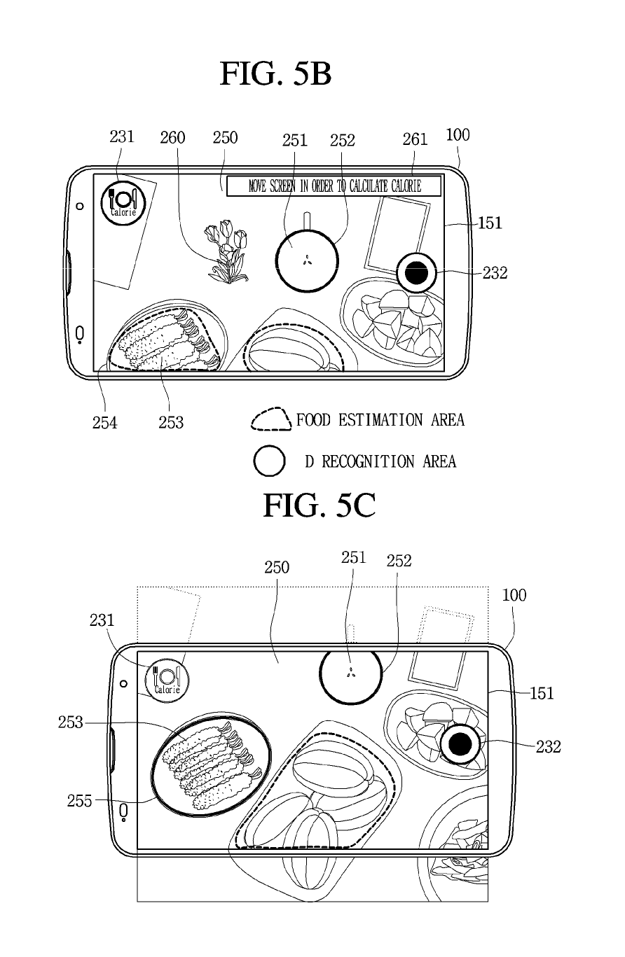

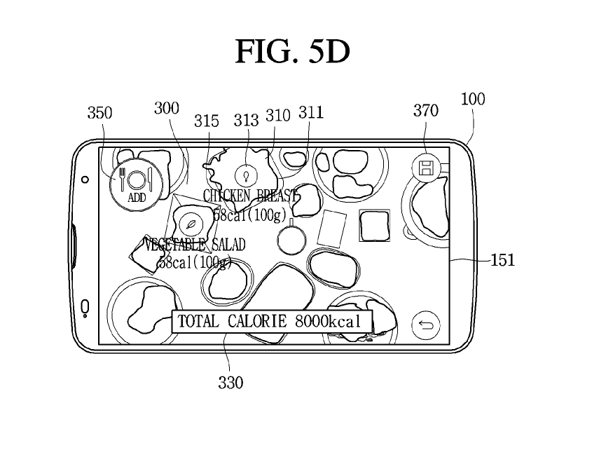

FIGS. 5a to 5d are diagrams illustrating a process of automatically food items included in a first preview image according to an embodiment of the present invention.

Referring to FIG. 5a, the display unit 151 displays the first preview image 250. In FIG. 5, assume that the mobile terminal 100 operates in the image capturing mode. When a request for selecting the calorie icon 231 is received, the control unit 180 may control the mobile terminal 100 to operate in the calorie recognition mode, as shown in FIG. 5b. The calorie recognition mode may be performed on the assumption that the image capturing mode is maintained. The control unit 180 may switch the image capturing icon 222 to the indicator 232 as the mobile terminal 100 operates in the calorie recognition mode. The indicator 232 may indicate that the mobile terminal 100 operates in the calorie recognition mode. The control unit 180 may recognize the food item 251 and the food estimation item 253 included in the first preview image 250. The control unit 180 may more accurately recognize the food item 251 using the first preview image enlarged according to input of enlarging the first preview image 250. The control unit 180 may display an area occupied by the food item 251 using a first identification line 252 having a first color and display an area occupied by the food estimation item 253 using a second identification line having a second color. The first identification line 252 may be a solid line and the second identification line 254 may be a dotted line, but this is only an example. The control unit 180 may distinguishably display the food item 251 recognized as a food and the food estimation item 253 estimated as a food. The food estimation item 253 may be an item, the full image of which is not included in the first preview image 250, that is, an item, a partial image of which is cut off. The control unit 180 may display a message window 261 for guiding screen movement in order to acquire accurate information on the food estimation item 253 through the display unit 151. Specifically, the control unit 180 may display a message window 261 in order to accurately grasp the calorie of the food estimation item 253.

The user may move the screen of the mobile terminal 100 to accurately recognize the food estimation item 253 estimated as a food. That is, when the full image of the food estimation item 253 is not displayed in the first preview image 250, the user may move the mobile terminal 100 such that the full image of the food estimation item 253 is displayed in the first preview image. In addition, the control unit 180 may acquire the 3D image of the food item according to tilting of the mobile terminal 100 and grasp the amount of food item through the 3D image. The amount of the food item may be used to calculate the food item later.

Referring to FIG. 5c, the food estimation item 253, the part of which is cut off in FIG. 5b, is recognized as a food. That is, the user may move the mobile terminal 100 such that the full area of the food estimation item 253 is included in the first preview image. The control unit 180 may recognize the full image of the food estimation item 253 and thus accurately acquire the information on the food estimation item 253. At this same time, the control unit 180 may change the second identification line 254 having the second color to the identification line 255 having the first color.

Meanwhile, a food may not be recognized as a food item or a food estimation item. In this case, as described with reference to FIGS. 4a to 4c, the unrecognized food may be recognized through food area designation input. In another embodiment, if a food is not recognized as a food item or a food estimation item in the first preview image 250, the calorie of the unrecognized item may be measured through various selection items. This will be described later.

When input of ending the calorie recognition mode is received, the control unit 180 may display information on each of the recognized food items through the display unit 151, as shown in FIG. 5d. Referring to FIG. 5d, the display unit 151 displays an analyzed image 300 for providing information on the recognized food items. In one embodiment, the control unit 180 may display the analyzed image 300 in response to input of ending the calorie recognition mode. Input of ending the calorie recognition mode may be input of selecting the indicator 232 shown in FIGS. 5a to 5c.

The analyzed image 300 may include the recognized food items, information on each food item, total calorie information 330 of the recognized food items, a food recognition addition icon 350 and a storage icon 370. For example, the recognized food item 310, the identification line 311 indicating the border line of the area occupied by the food item 310, a category icon 313 indicating the category of the food item 310 and detailed information 315 of the food item 310 may be displayed in the analyzed image 300. The category icon 313 may indicate the type of the recognized food item 310. The detailed information 315 of the food item 310 may include one or more of the name of the food item 310, the calorie of the food item 310, the amount (grams) of the food item 310. The user may select the category icon 313 to change the category or detailed information of the food item 310.

The total calorie information 330 may provide a result of summing the total calorie of the plurality of food items recognized in the first preview image 250 through the process of FIGS. 5a to 5c. The user may confirm the total calorie of the foods to be ingested thereby through the total calorie information 330.

The food recognition addition icon 350 may be an icon for recognizing the unrecognized food item among the food items included in the analyzed image 300 and adding the calorie of the food item to the total calorie information 330. When the request for selecting the food recognition addition icon 350 is received, the unrecognized food item may be additionally recognized according to the process described with reference to FIGS. 4b and 4c. The calorie of the recognized food item may be added to the total calorie information 330.

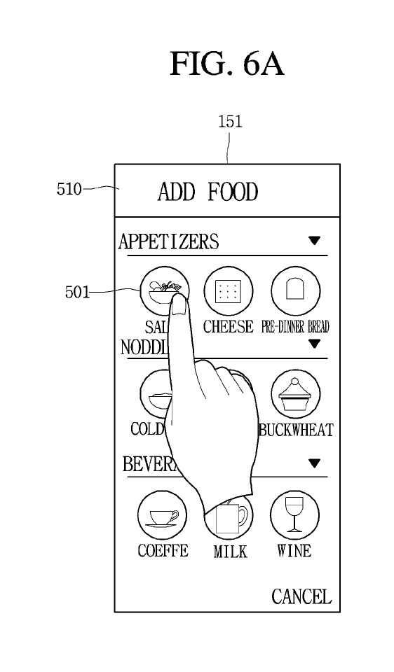

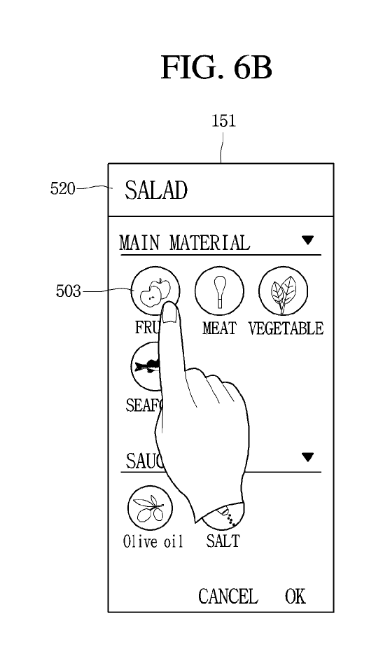

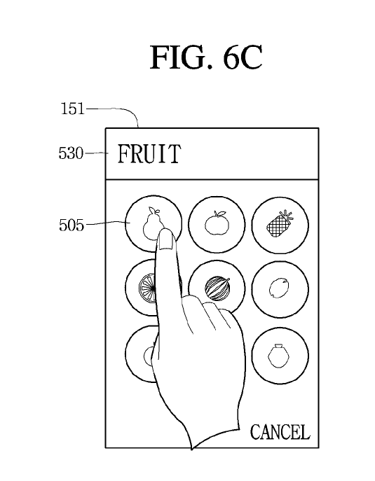

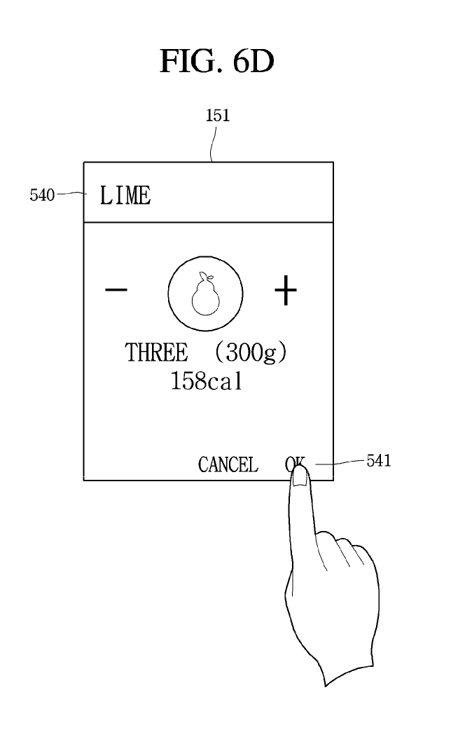

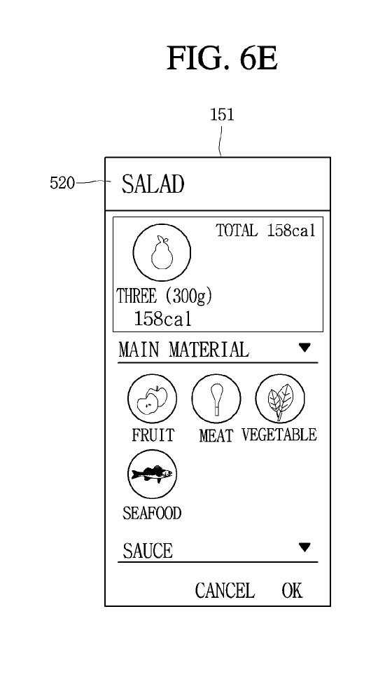

In another embodiment, various selection items may be provided through the food addition icon 350 to perform calorie measurement. This will be described with reference to FIGS. 6a to 6e.

FIGS. 6a to 6e are diagrams illustrating a process of manually recognizing a food included in a first preview image to perform calorie measurement according to another embodiment of the present invention.

In FIGS. 6a to 6e, the unrecognized food item is a salad item.

When the request for selecting the food recognition addition icon 350 shown in FIG. 5d is received, the control unit 180 may control the display unit 151 to display a manual food addition window 510 for manually adding a food item, as shown in FIG. 6a. The manual food addition window 510 may include high-level category items (appetizers, noodles and beverages) for classifying high-level categories of foods. When a request for selecting a salad item 501 in an appetizer item is received, the control unit 180 may display a material provision window 520 for selecting the materials of the selected salad item 501 through the display unit 151, as shown in FIG. 6b. The material provision window 520 may include a main material item and a sauce item. When a request for selecting a fruit item 530 in a main material item is received, the control unit 180 may display a fruit type provision window 530 for selecting the type of fruit through the display unit 151, as shown in FIG. 6c.

When a lime item 505 is selected in the fruit type provision window 530, the control unit 180 may display a line information provision window 540 for selecting the number of limes, as shown in FIG. 6d. The calorie may be changed according to the number of limes. When the number of limes is selected and an OK button 541 is selected, the control unit 180 may display the material provision window 520 again, as shown in FIG. 6e. In the case of the salad, since materials other than fruit may be further included, as the user selects other main materials and a sauce, it is possible to accurately measure the calorie of the salad item.

FIGS. 6a to 6e may be used even when a food is eaten at a restaurant in which the mobile terminal 100 is located. For example, when the mobile terminal 100 is located in the restaurant, the control unit 180 may acquire a menu provided by the restaurant. That is, the control unit 180 may acquire the menu including foods provided by the restaurant from a server of the restaurant through short-range wireless communication. The control unit 180 may display a manual food addition window 510 shown in FIG. 6a based on the acquired menu. The user may select a food to be ordered thereby from among the foods provided by the restaurant through the process of FIGS. 6a to 6e and grasp the calorie of the selected food in advance.

FIG. 2 will be described again.

After acquiring the information on the recognized food item or food estimation item, the control unit 180 may receive input of ending the calorie recognition mode (S119), and display an analyzed image including information on each food item in response to the received input (S121).

That is, as shown in FIG. 5, the control unit 180 may display the analyzed image 300 including the information on the recognized food information according to input of ending the calorie recognition mode.

The embodiment of displaying the analyzed image 300 is the same as for the food item recognized through manual input as in the embodiment of FIGS. 4a to 4c.

The control unit 180 displays a memo window according to a request for selecting a specific food item included in the displayed analyzed image (S123), and receives additional information in the memo window and stores the additional information in the memory 170 (S125). In one embodiment, the memo window may be a window for inputting information on the selected food item. This will be described with reference to the following drawings.

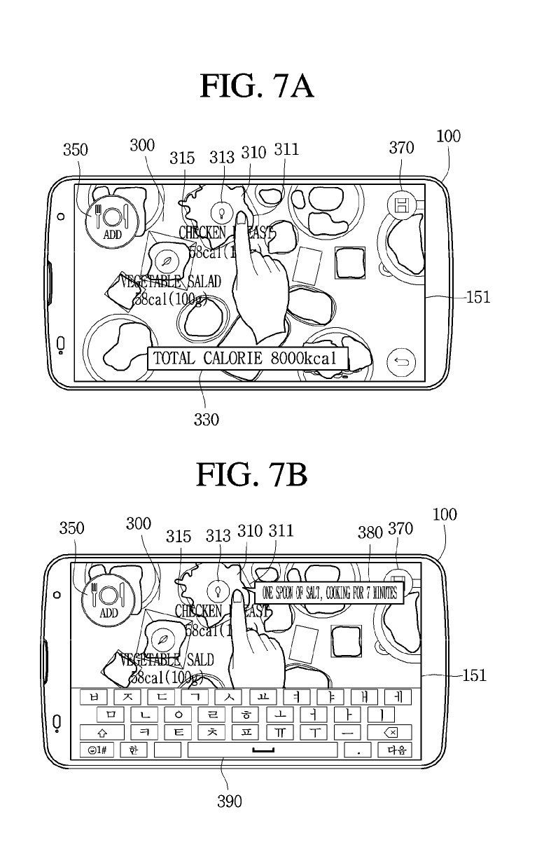

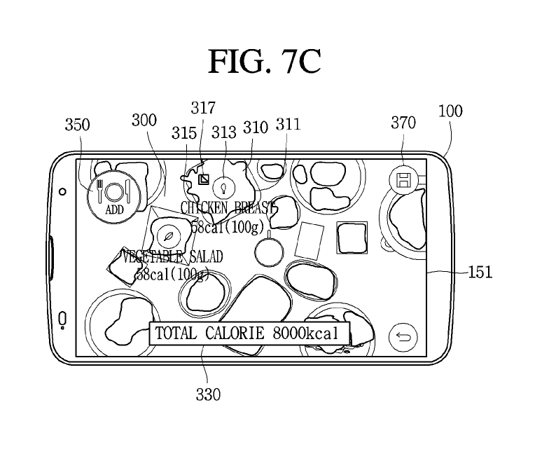

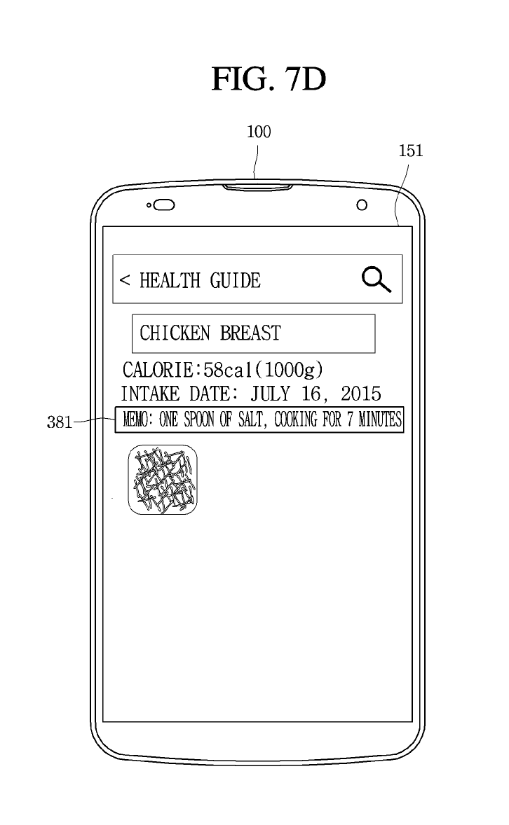

FIGS. 7a to 7d are diagrams illustrating a memo function for manually inputting additional information with respect to a recognized food according to an embodiment of the present invention.

Referring to FIG. 7a, a request for selecting (long touch) a specific food item 310 for a predetermined time or more in the analyzed image 300 may be received. Therefore, the control unit 180 may display a memo window 380 for inputting additional information of the selected food item 310 and a text input window 390, as shown in FIG. 7b. When a memo is input in the memo window 380 through the text input window 390, the control unit 180 may store the input memory in the memory 170 in correspondence with the food item 310. The input memo may include a recipe of the food item 310, the name of the restaurant, etc. When the input memo is stored, the control unit 180 may display a memo icon 317 indicating that the memo is added to the food item 310 on the food item 310, as shown in FIG. 7c. The control unit 180 may display the input memo according to a request for selecting the memo icon 317. The user may edit the input memo.

FIG. 7d shows an execution screen of the eating-habit management application. When the name of the food item 310 that inputs a memo in the search window of the execution screen, the control unit 180 may display a pre-stored memo 381, the intake amount of the food, and the intake time of the food.

FIG. 2 will be described again.

The control unit 180 senses that the food intake situation of the user ends (S127), and the control unit 180 outputs an eating-habit management notice (S129) upon sensing that the food intake situation ends.

In one embodiment, the control unit 180 may sense the food intake end situation based on a voice command input to the microphone or sound related to food intake. For example, the control unit 180 may sense that the food intake situation ends when the voice command “I enjoyed this meal” or “Please remove it” is input to the microphone 122, it is sensed that the food intake situation ends. As another example, the control unit 180 may sense that the food intake situation end, when sound of hitting dishes is not input to the microphone 122 for a predetermined time.

In another embodiment, the control unit 180 may sense the food intake situation of the user using the camera 121. For example, when the food intake operation of the user is not sensed through the camera 121 for a predetermined time, the control unit 180 may confirm that the food intake situation ends.

In another embodiment, the control unit 180 may sense that the food intake situation of the user ends when a meal end time set by the user arrives. For example, if the meal time set by the user is 8:30 AM to 9:00 AM, the control unit 180 may sense that the food intake situation of the user ends when the current time is 9:00 AM.

In another embodiment, the control unit 180 may sense that the food intake situation ends when meal end input is received in an eating-habit management notice bar displayed in a status bar. Step S127 will be described with reference to the following drawings.

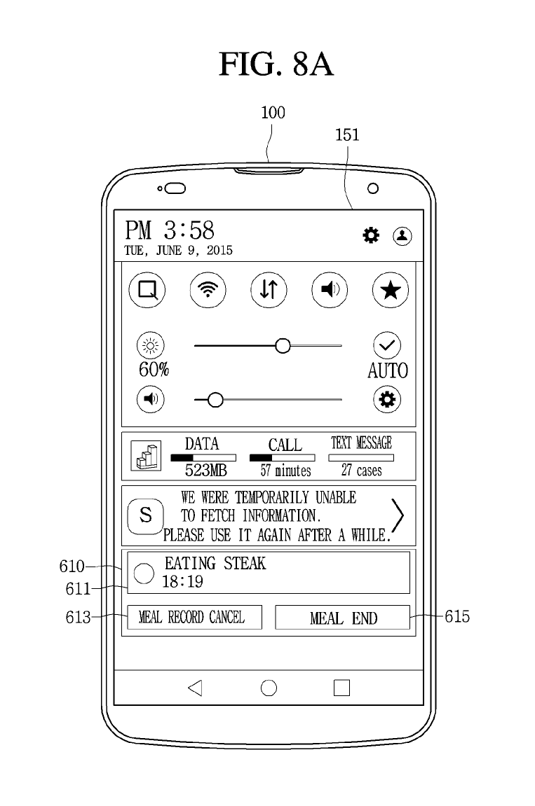

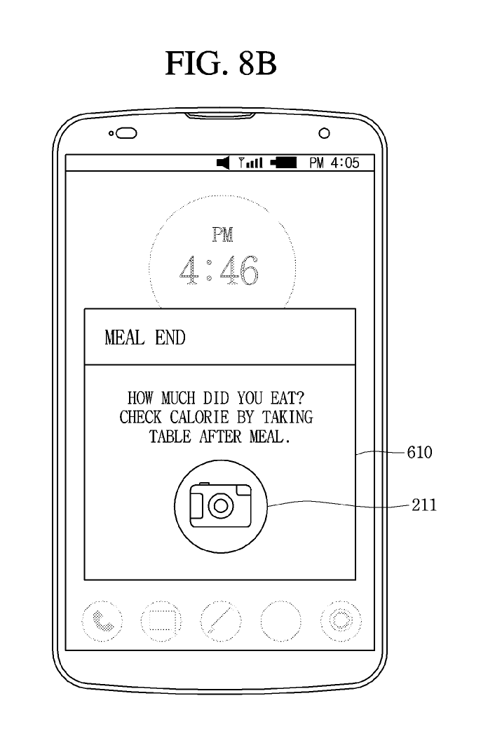

FIGS. 8a and 8b are diagrams illustrating an example of an eating-habit management notice according to another embodiment of the present invention.

Referring to FIG. 8a, the mobile terminal 100 displays a status bar for providing information on the status of the mobile terminal 100 through the display unit 151. The status bar may include an eating-habit management notice bar 610 for eating-habit management. The eating-habit management notice bar 610 may be displayed in a state of executing the eating-habit management application. The eating-habit management notice bar 610 may indicate that the user is currently in the food intake situation. The eating-habit management notice bar 610 may include a during-meal item 611 indicating that the user is eating, a cancellation item 613 for canceling a meal record and a meal end item 615 indicating that user's meal ends. When a request for selecting the during-meal item 611 is received, the control unit 180 may display the analyzed image 300 of FIG. 5d. The user may select a food addition icon 350 to add a food item to be recognized. When a request for selecting a meal end item 615 is received, the control unit 180 may sense that the food intake situation of the user ends.

Upon sensing that the food intake situation ends, the control unit 180 may display a popup window 610 for providing an eating-habit management notice through the display unit 151, as shown in FIG. 8b. The popup window 610 may provide the calorie of the food ingested by the user after meals. The popup window 610 may include guide text <After meals, check a calorie by shooting a table> and a camera icon 211 having a camera shape. The camera icon 211 may be an icon for reentering the operation mode of the mobile terminal 100 to the calorie recognition mode.

FIG. 2 will be described again.

The control unit 180 displays a second preview image after reentering the calorie recognition mode (S131). In one embodiment, the second preview image may be an image reflecting that the food intake situation of the user ends as compared to the first preview image.

The control unit 180 acquires the intake calorie based on the intake amount of each food item included in the second preview image (S133) and displays a total intake calorie after meals (S135). In one embodiment, the control unit 180 may calculate a total calorie ingested by the user using a difference between the total calorie acquired through the first preview image and the total calorie acquired through the second preview image. Steps S131 to S135 will be described with reference to the following drawings.

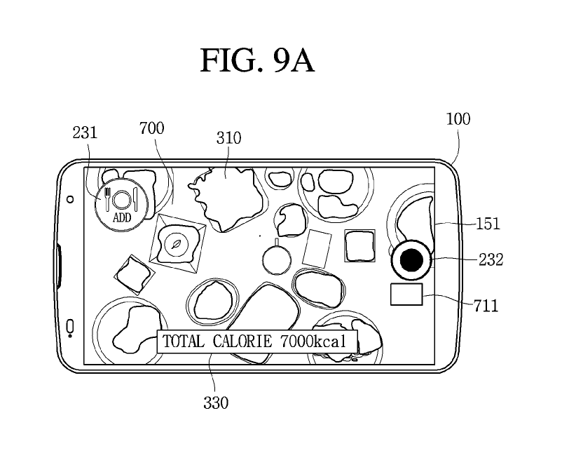

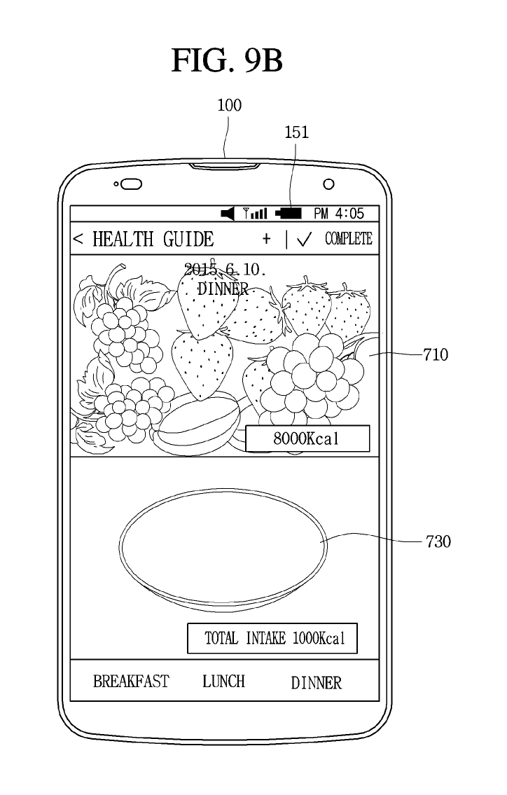

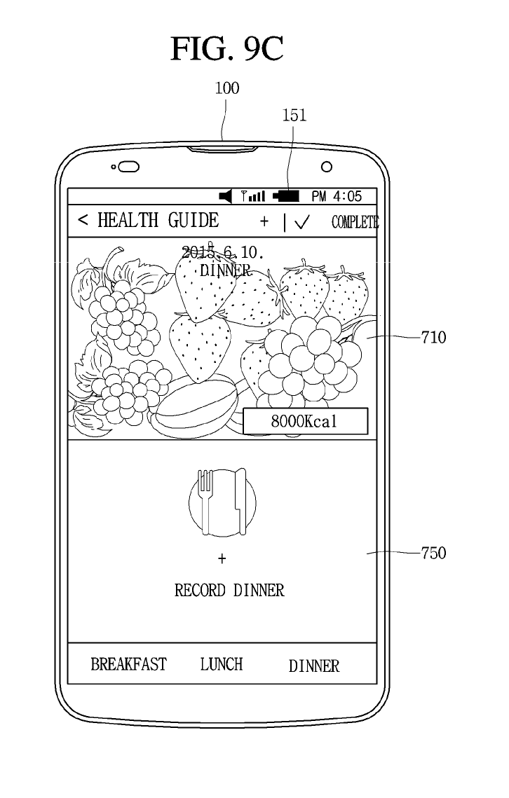

FIGS. 9a to 9c are diagrams illustrating a process of providing a calorie ingested by a user after the food intake situation of the user ends according to an embodiment of the present invention.

Upon receiving a request for selecting the image capturing icon 221 in FIG. 8b, the control unit 180 may control the mobile terminal 100 to enter the calorie recognition mode and display the second preview image 700 reflecting that the food intake situation ends through the display unit 151, as shown in FIG. 9a. The second preview image 700 may be acquired through the camera. On the second preview image 700, a calorie icon 231, an indicator 232 indicating operation in the calorie recognition mode and a result provision icon 711 may be further displayed. The second preview image 700 may display the states of the intake items of the plurality of food items. The control unit 180 may recognize the plurality of food items included in the second preview image 700 according to a request for selecting the calorie icon 231. The result provision icon 711 may provide the calorie ingested by the user based on the calories of the recognized food items.

The control unit 180 may recognize the plurality of food items included in the second preview image 700, as described with reference to FIGS. 5a to 5c. The control unit 180 may acquire the amount of food ingested by the user through the recognized food items and calculate the calorie of the food ingested by the user based on the acquired amount of food. For example, the control unit 180 may calculate the calorie based on the amount of food item 310 included in the second preview image 700. By this method, the control unit 180 may calculate the calorie corresponding to each of the plurality of food items included in the second preview image 700. The control unit 180 may acquire the analyzed image of the second preview image 700, as shown in FIG. 5d. Therefore, the control unit 180 may acquire the total calorie (7000 kcal) of the plurality of food items included in the second preview image 700.

The control unit 180 may acquire a difference (1000 Kcal) between the total calorie (80000 Kcal) of the plurality of food items acquired through the analyzed image 300 of the first preview image 250 and the total calorie (7000 Kcal) of the plurality of food items acquired through the second preview image 700. This may be the calorie ingested by the user.

Upon receiving a request for selecting the result provision icon 711, the control unit 180 may display information 710 on the calorie before meals and information 730 on the calorie after meals through the display unit 151, as shown in FIG. 9b. The information 730 on the calorie after meals may include the calorie (1000 Kcal) ingested by the user. The user may confirm the calorie ingested by the user in real time by only capturing the images of the foods before and after meals. Therefore, the user may plan the eating habits thereof by reflecting the calorie intake amount thereof.

Meanwhile, when a request for selecting the result provision icon 711 is received and the second preview image 700 is not captured, the control unit 180 may display a manual input window 750 for manual input, as shown in FIG. 9c. The user may input information on the food ingested thereby through the manual input window and receive a calorie intake result.

According to another embodiment of the present invention, a food image captured through the mobile terminal 100 may be stored in the memory 170 in correspondence with the calorie. In addition, upon sharing the food image with another user, the stored calorie may be automatically added and shared.

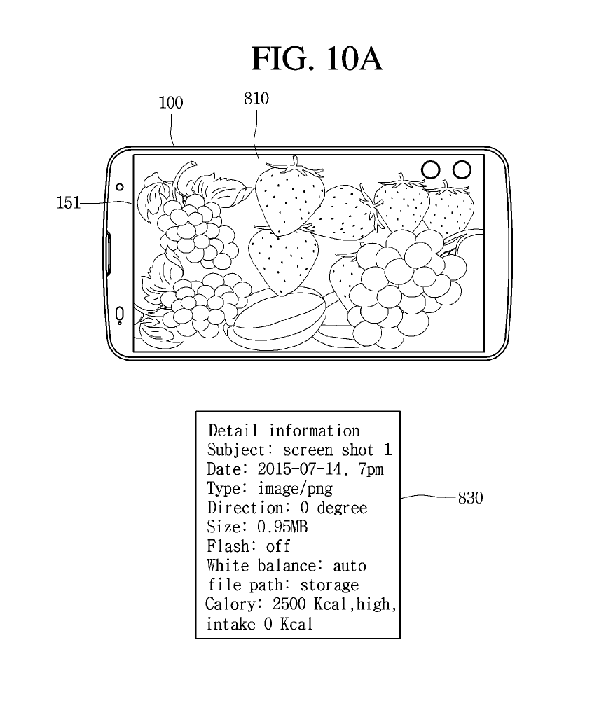

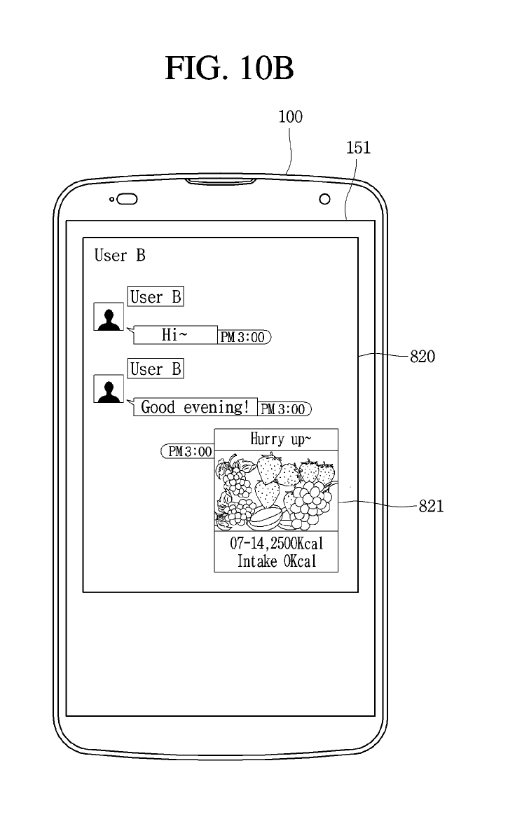

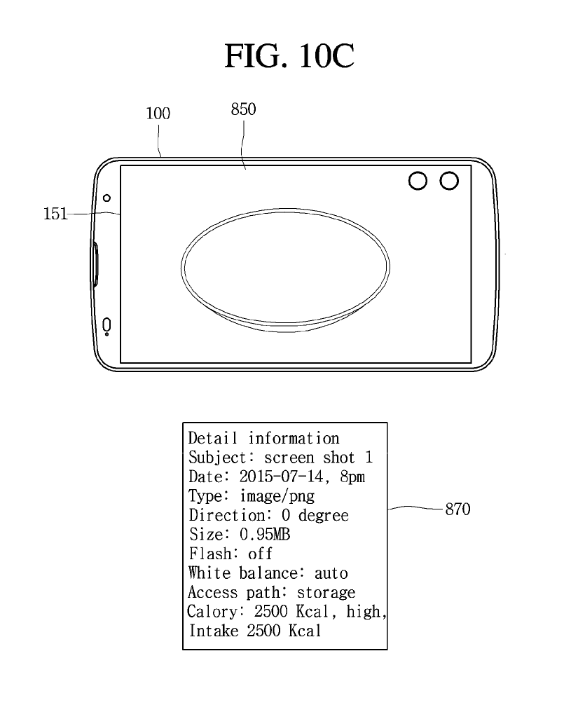

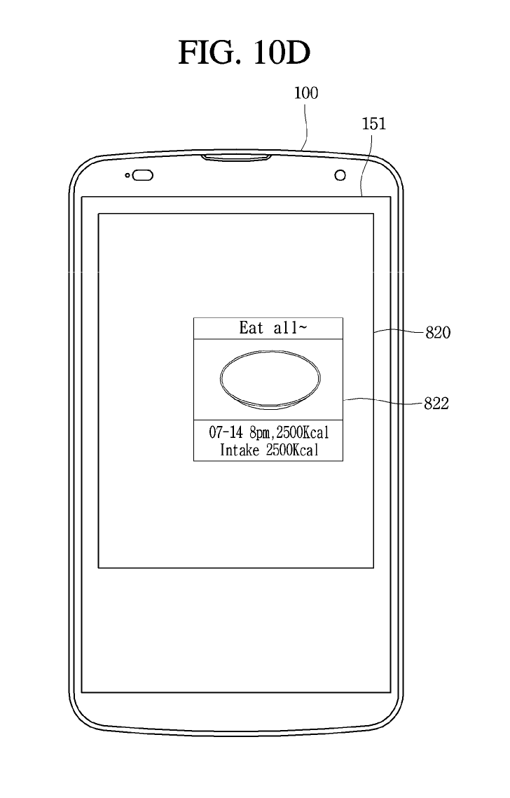

FIGS. 10a to 10d are diagrams illustrating a process of automatically adding calorie information upon sharing a captured food image according to an embodiment of the present invention.

Referring to FIG. 10a, the display unit 151 of the mobile terminal 100 displays a food image 810. Assume that the mobile terminal 100 recognizes the food items included in the food image 810 and acquires the calorie of each of the recognized food items. The food image 810 may be captured through the camera 121. The control unit 180 may store the food image 810 in the memory 170 in correspondence with the information on the food items included in the food image 810. For example, the food image 810 is stored in the memory 170 to correspond to detailed information 830 thereof. The detailed information 830 may include the title of the food image 810, the capturing time, the type of the image, the angle of the image, the size of the image, flashlight on/off, white balance, storage path, and the calorie information of the food item included in the food image 810. When the mobile terminal 100 shares the food image 810 with another mobile terminal, some of the detailed image 830 corresponding to the food image 810 may also be transmitted.

Referring to FIG. 10b, the display unit 151 displays an execution window 820 of a mobile message application for providing a service for sharing media content. When the user of the mobile terminal 100 transmits content 821 including the food image through the execution window 820, the calorie information of the food item included in the food image may be transmitted. The calorie information may include the total calorie (2500 Kcal) of the food items and the intake calorie (0 Kcal) of the user.

The user may share the food image 850 after eating foods. Referring to FIG. 10c, the mobile terminal 100 may capture the food image 850 after eating the foods through the camera 121. The control unit 180 may confirm the intake amount of food item through the captured food image 850 and acquire the calorie ingested by the user based on the intake amount of food item. The food image 850 is stored in the memory 170 to correspond to detailed information 870 thereof. The detailed information 870 may include the title of the food image 850, the capturing time, the type of the image, the angle of the image, the size of the image, flashlight on/off, white balance, storage path, and the calorie information of the food item included in the food image 850.

Referring to FIG. 10d, the display unit 151 displays an execution window 820 of a mobile message application for providing a service for sharing media content. When the user of the mobile terminal 100 transmits content 822 including the food image through the execution window 820, the calorie information of the food item included in the food image may be transmitted. The calorie information may include the total calorie (2500 Kcal) of the food items and the calorie (2500 Kcal) ingested by the user.

Upon sharing the food image before meals and the food image after meals with another user, information on the intake calorie may be automatically added.

According to another embodiment of the present invention, the mobile terminal 100 may provide the calorie ingested by the user with passage of time.

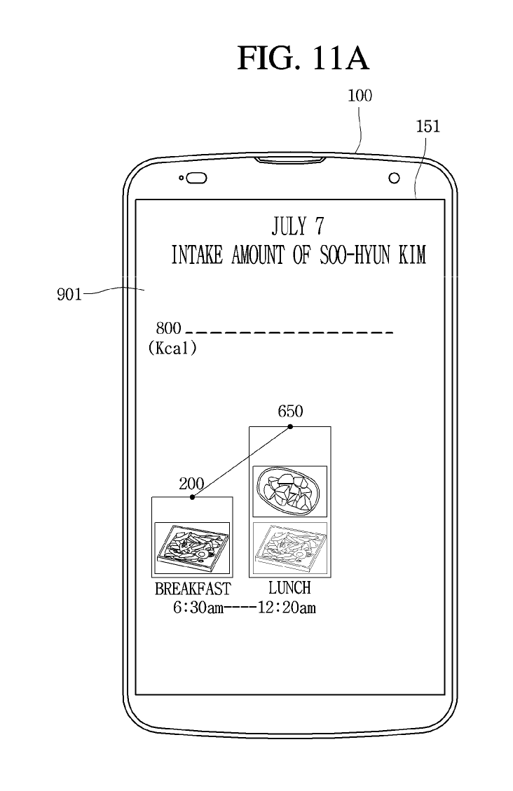

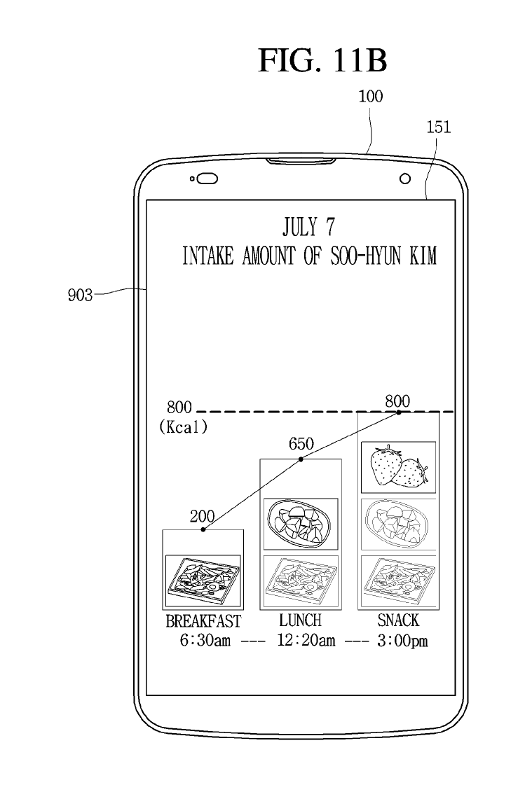

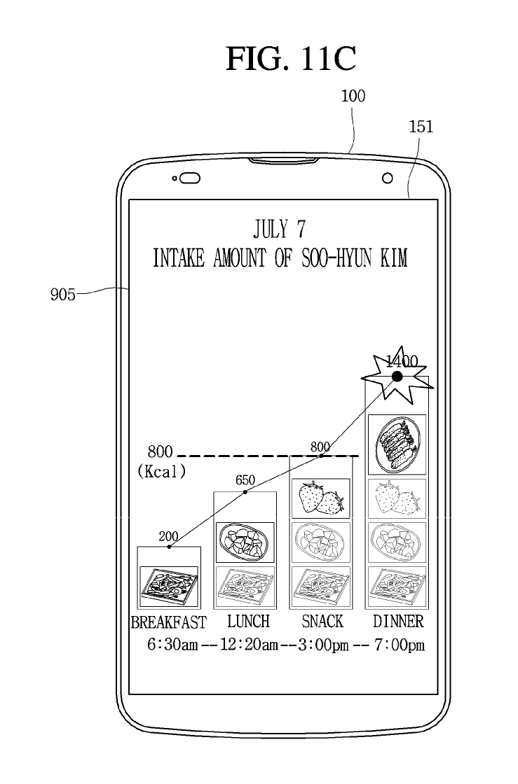

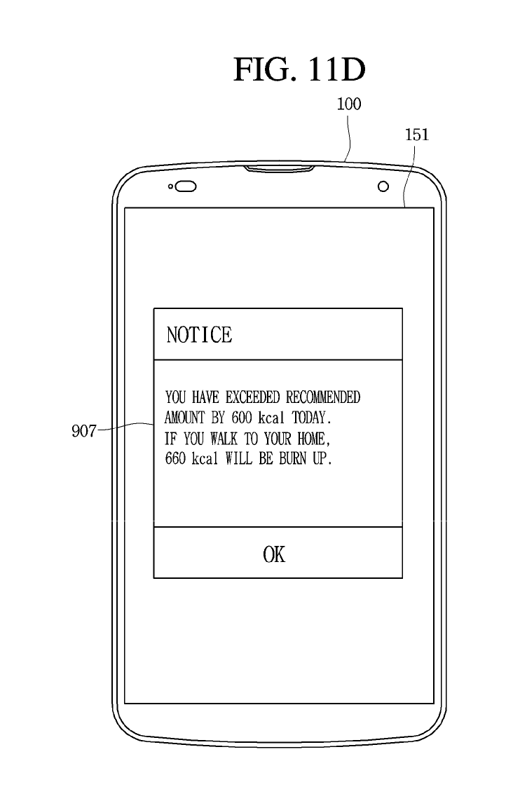

FIGS. 11a to 11d are diagrams illustrating an embodiment of providing a calorie ingested by a user with the passage of time.

In FIGS. 11a to 11d, assume that a recommended daily intake calorie is 800 Kcal. In addition, the following graphs may be generated based on the calories acquired through the embodiments of FIGS. 4a to 4c or FIGS. 5a to 5d.

Referring to FIG. 11a, the display unit 151 of the mobile terminal 100 displays a calorie intake amount graph including a calorie (200 Kcal) ingested for breakfast of a specific date and a calorie (650 Kcal) obtained by summing the calorie (200 Kcal) ingested for the breakfast and a calorie (450 Kcal) ingested for lunch. The calorie intake amount graph 901 may include an image of a food ingested by the user at a corresponding time. Referring to FIG. 11b, a calorie intake amount graph 903 reflecting the calorie (150 Kcal) of a snack ingested by the user at 3:00 PM is shown. That is, the recommended daily calorie intake amount of 800 Kcal has been already reached. Referring to FIG. 11c, the display unit 151 displays a calorie intake amount graph 905 reflecting a calorie (600 Kcal) ingested for a dinner. The calorie ingested by the user for one day is 1400 Kcal and thus exceeds the recommended calorie intake amount of 800 Kcal. In this case, the control unit 180 may display a popup window 907 indicating that the calorie ingested by the user exceeds the recommended calorie intake amount of today and recommending a workout through the display unit 151, as shown in FIG. 11d.

According to one embodiment of the present invention, the mobile terminal 100 may guide the amount of food to be ingested such that the daily calorie intake amount of the user does not exceed the recommended calorie intake amount.

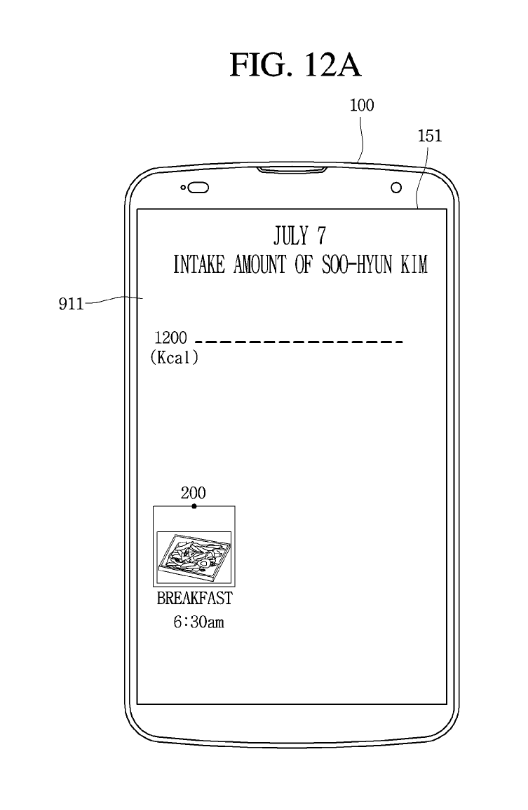

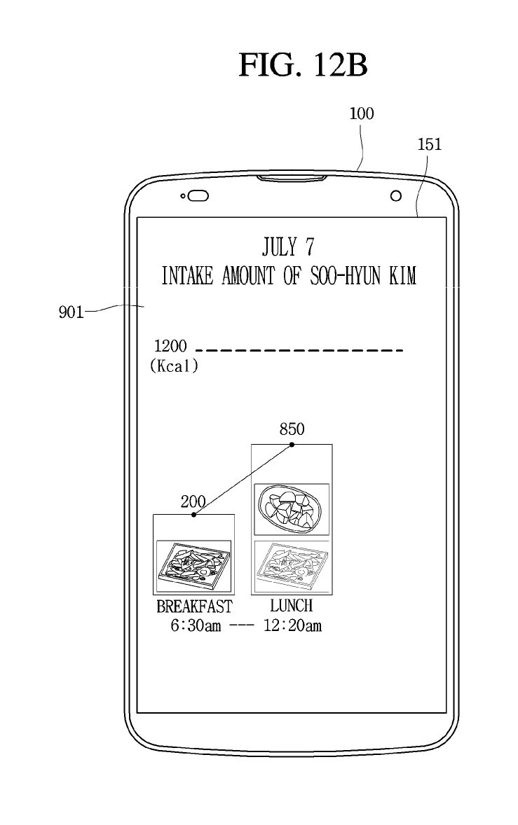

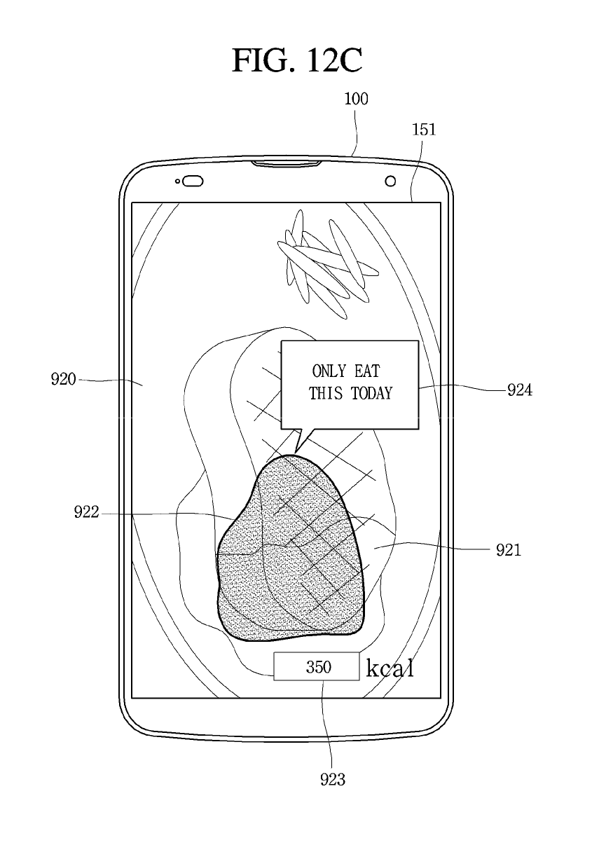

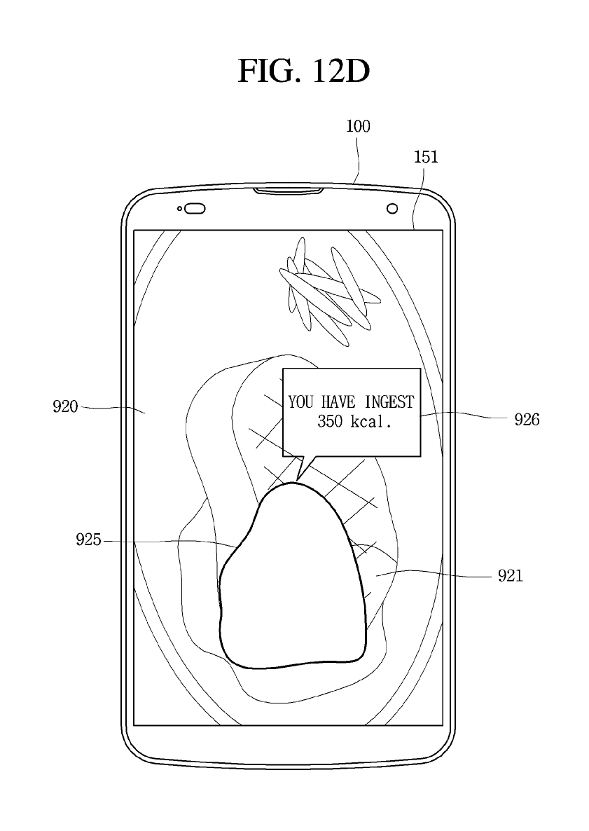

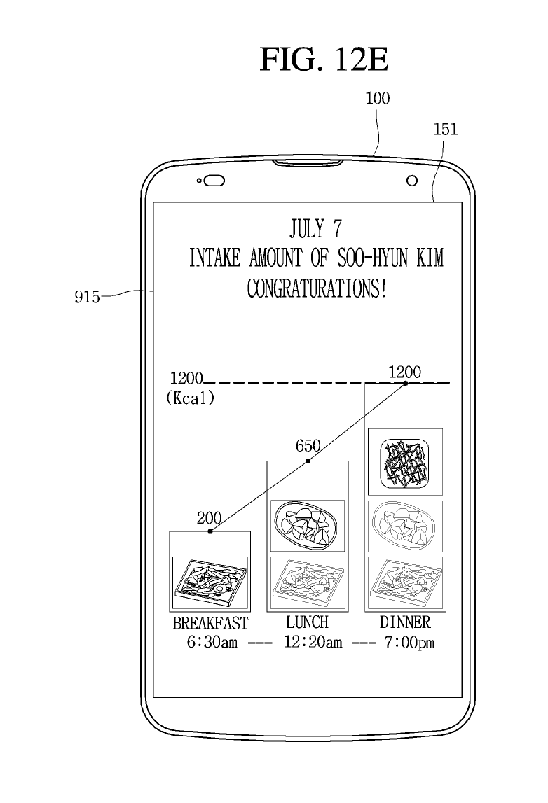

FIGS. 12a to 12e are diagrams illustrating a process of guiding the amount of foods to be ingested such that the daily calorie intake amount of a user does not exceed a recommended calorie intake amount according to an embodiment of the present invention.

In FIGS. 12a to 12e, assume that a recommended daily calorie intake amount is 120 Kcal. In addition, the following calorie intake amount graph may be generated based on the calories acquired through the embodiments of FIGS. 4a to 4c or FIGS. 5a to 5d.

Referring to FIG. 12a, the display unit 151 displays a calorie intake amount graph 911 including a calorie (200 Kcal) ingested by the user for breakfast. Referring to FIG. 12b, a calorie intake amount graph 913 including a calorie (650 Kcal) obtained by summing the calorie (200 Kcal) ingested by the user for breakfast and the calorie (450 Kcal) ingested by the user for lunch is shown. 350 Kcal remains until the recommended calorie intake amount (1200 Kcal) is reached.

Referring to FIG. 12c, the display unit 151 displays a preview image 915 including a food item 921 to be ingested by the user for dinner upon entering the calorie recognition mode. The control unit 180 may recognize the food item 921 and acquire the calorie of the food item 921. Since 350 Kcal remains until the recommended calorie intake amount is reached, the control unit 180 may extract only a partial area 922 corresponding to 350 Kcal from the entire area of the recognized food item 921. The partial area 922 of the food item 921 may be displayed to be distinguished from the remaining area. A calorie value 923 corresponding to the partial area 922 may be displayed in the partial area 922. In addition, text 924 for recommending the user to eat only some of the food item 921 not to exceed the recommended daily calorie intake amount may be displayed at one side of the partial area 922.

As the user reenters the calorie recognition mode after dinner, the display unit 151 may display a preview image 930 including a food item 921. An area 925 ingested by the user of the entire area of the food item 921 may be distinguishably displayed. Text 926 including the calorie (350 Kcal) corresponding to the area 925 ingested by the user may be displayed in order to guide the calorie ingested by the user.

Referring to FIG. 12e, a calorie intake amount graph 915 reflecting a calorie (350 Kcal) ingested for dinner is displayed. Since a daily calorie intake amount does not exceed the recommended calorie intake amount, text indicating target achievement may be further displayed.

According to the embodiment of the present invention, it is possible to acquire the calorie ingested by the user in real time and to improve the eating habits of the user by specifying the amount of foods to be ingested by the user.

According to another embodiment of the present invention, the mobile terminal 100 may provide information on the calorie ingested by the user for one day at a glance.

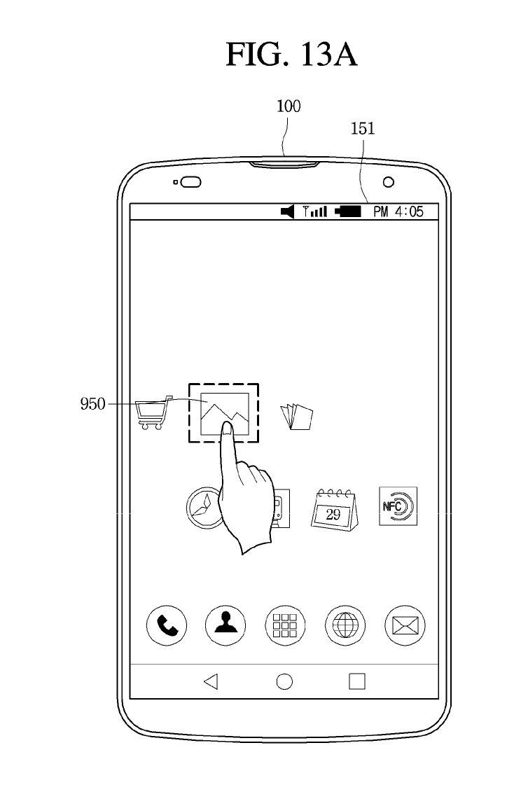

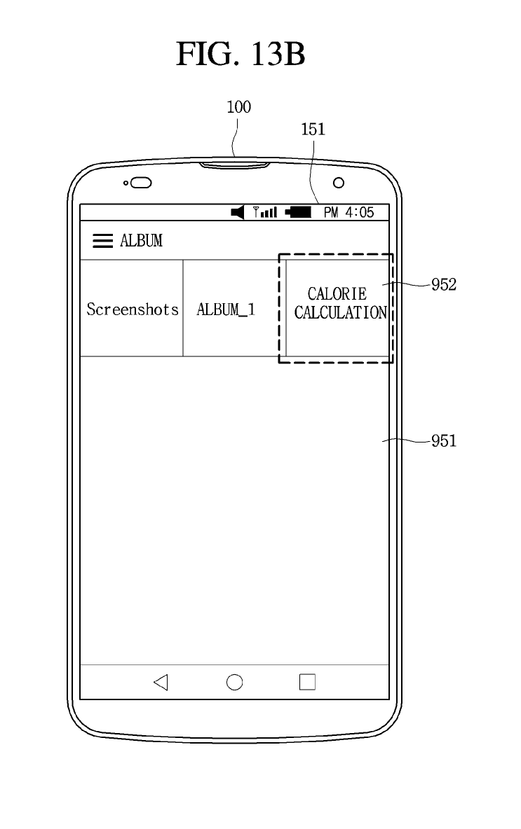

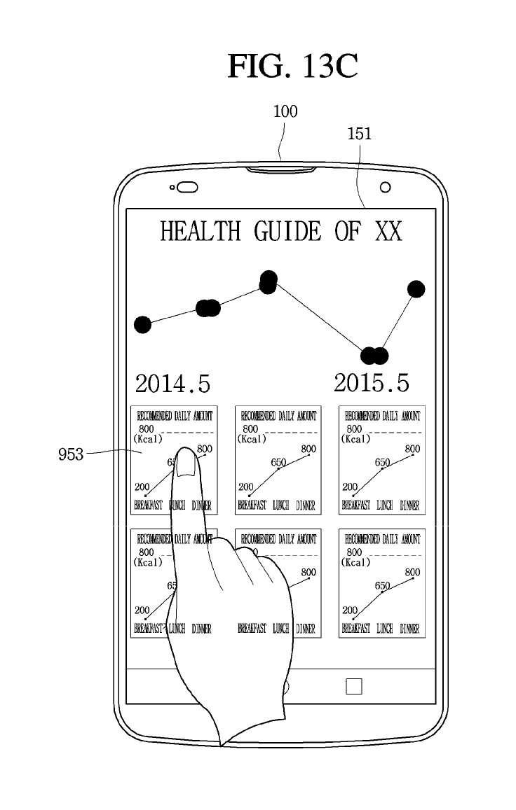

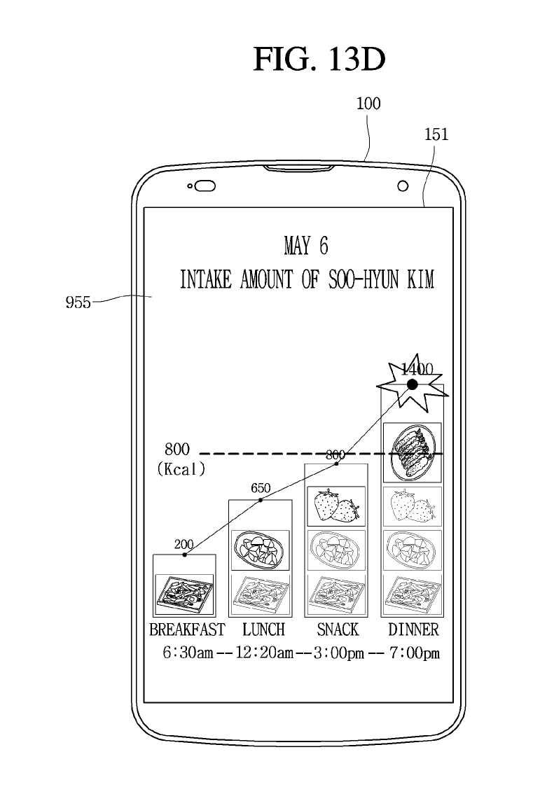

FIGS. 13a to 13d are diagrams illustrating a process of providing information on foods and calories ingested by a user every day at a glance according to an embodiment of the present invention.

Referring to FIG. 13a, the display unit 151 displays a home screen. On the home screen, a gallery app icon 950 corresponding to a gallery application for providing stored content is displayed. The control unit 180 may display a stored content history 951 according to a request for selecting the gallery app icon 950, as shown in FIG. 13b. The content history 951 may include a calorie folder 952 for providing the food and calorie ingested by the user by date. The control unit 180 may display a calorie information provision item 953 for providing information on the food and calorie ingested by the user on a corresponding date according to a request for selecting the calorie folder 952, as shown in FIG. 13c. According to a request for selecting the calorie information provision item 953 on May 6, the control unit 180 may display a calorie intake amount graph 955 including foods ingested by the user on the corresponding date and a calorie ingested by the user with passage of time, as shown in FIG. 13d. The calorie intake amount graph 955 may provide a recommended calorie intake amount (800 Kcal) for one day and the amount of calories ingested by the user at a corresponding time. The user may easily grasp the amount of foods and calories ingested thereby per day at a glance. The user may plan the future eating habits by referring to the amount of foods and calories.

The invention can also be embodied as computer readable codes on a computer readable recording medium. The computer readable recording medium is any data storage device that can store data which can be thereafter read by a computer system. Examples of the computer readable recording medium include hard disk drives (HDDs), solid state disks (SSDs), silicon disk drives (SDDs), ROMs, RAMs, CD-ROMs, magnetic tapes, floppy disks, and optical data storage devices, and also include carrier waves (such as data transmission through the Internet). Furthermore, the computer may include the control unit 180 of the terminal. Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1.-16. (canceled)

17. A mobile terminal, comprising:
a microphone;
a camera;
a display; and
a controller configured to:
detect a food intake situation based on a sound inputted to the microphone,
operate the camera to be turned on according to detecting the food intake situation,
display, on the display, a first preview image including a food item captured through the camera,
acquire information on the food item, and
display, on the display, the information on the food item at an adjacent position of the food item.

18. The mobile terminal according to claim 17, wherein the controller detects the food intake situation using a voice command of a user or a sound of hitting dishes inputted to the microphone.

19. The mobile terminal according to claim 17, wherein the controller transmits the captured food item to a server, and acquires the information on the food item from the server.

20. The mobile terminal according to claim 17, wherein the information on the food item includes at least one of a name of the food item, a calorie of the food item or grams of the food item.

21. The mobile terminal according to claim 17, wherein the controller receives an input of designating an area occupied by the food item, and acquires the information on the food item according to the input.

22. The mobile terminal according to claim 21, wherein the controller displays, on the display, a second preview image if the controller detects that the food intake situation is finished and acquires a calorie of a remaining amount of the food item based on the remaining amount of the food item included in the second preview image.

23. The mobile terminal according to claim 22, wherein the controller acquires a calorie ingested by the user using a difference between the calorie of the food item acquired in response to the received input and the calorie of the remaining amount of the food item, and displays the calorie ingested by the user.

24. The mobile terminal according to claim 17, wherein the controller receives a request for selecting the food item, and display, on the display, a memo window for inputting additional information of the selected food item.

25. A mobile terminal, comprising:
a microphone;
a camera;
a display unit; and
a controller configured to:
detect a situation based on an audio inputted to the microphone,
operate the camera to be turned on according to detecting the situation,
display, on the display unit, a first preview image including a subject captured through the camera,
acquire information on the subject, and
display, on the display unit, the information on the subject at an adjacent position of the subject.

26. The mobile terminal according to claim 25, wherein the controller detects a food intake situation using the audio inputted to the microphone, and
wherein subject includes a food item.

27. The mobile terminal according to claim 26, wherein the controller detects the food intake situation using a voice command of a user or a sound of hitting dishes inputted to the microphone.

28. The mobile terminal according to claim 26, wherein the controller transmits the captured food item to a server, and acquires the information on the food item from the server.

29. The mobile terminal according to claim 26, wherein the information on the food item includes at least one of a name of the food item, a calorie of the food item or grams of the food item.

30. The mobile terminal according to claim 26, wherein the controller receives an input of designating an area occupied by the food item, and acquires the information on the food item according to the input.

31. The mobile terminal according to claim 30, wherein the controller displays, on the display, a second preview image if the controller detects that the food intake situation is finished and acquires a calorie of a remaining amount of the food item based on the remaining amount of the food item included in the second preview image.

32. The mobile terminal according to claim 31, wherein the controller acquires a calorie ingested by the user using a difference between the calorie of the food item acquired in response to the received input and the calorie of the remaining amount of the food item, and displays the calorie ingested by the user.

33. The mobile terminal according to claim 26, wherein the controller receives a request for selecting the food item, and display, on the display, a memo window for inputting additional information of the selected food item.
